(19) United States
(12) Patent Application Publication
(10) Pub. No.: US 2005/0226863 A1
(43) Pub. Date: Oct. 13, 2005

(54) Single-domain antibodies and uses thereof

(75) Inventors: David W. Colby, Cambridge, MA (US); K. Dane Wittrup, Chestnut Hill, MA (US); Vernon M. Ingram, Cambridge, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Massachusetts Institute of Technology, Cambridge, MA (US)

(21) Appl. No.: 10/995,074

(22) Filed: Nov. 22, 2004

Related U.S. Application Data
(60) Provisional application No. 60/523,842, filed on Nov. 20, 2003.

(51) Int. Cl. A61K 39/395; C07K 16/44

(52) U.S. Cl. 424/132.1; 530/387.3

(57) ABSTRACT

The invention relates in part to methods of making single-domain antibodies and methods of using single-domain antibodies to diagnose and treat disease. The invention also relates to methods and products for modulating target protein activity, including methods to inhibit huntingtin protein aggregation in Huntington's disease. The invention also includes methods and compounds for identifying pharmaceutical agents for preventing and treating diseases and for monitoring the efficacy of treatments for target protein-associated diseases.

### RELATED APPLICATION

This application claims priority under 35 U.S.C. §119 from U.S. provisional application Ser. No. 60/523,842, filed Nov. 20, 2003, the contents of which is incorporated herein in its entirety.

### FIELD OF THE INVENTION

The invention relates in part to methods of making single-domain antibodies and methods of using single-domain antibodies to diagnose and treat disease. The invention also relates to methods and products for modulating target protein activity, including methods to inhibit huntingtin protein aggregation in Huntington's disease. The invention also includes methods and compounds for identifying pharmaceutical agents for preventing and treating diseases.

### BACKGROUND OF THE INVENTION

Strategies for making antibodies for diagnostics and therapeutics applications are of growing importance in the medical arts. One area of increasing interest pertains to the intracellular use of antibody fragments, referred to as intrabodies. (For review see: Stocks, M. R. Drug Disc. Today Vol 9, No. 22 Nov. 2004). These intracellular fragments, called intrabodies, are believed to have great promise in medicine and are the focus of much research to design functional and efficient intrabody-development strategies.

Numerous attempts have been made to improve antibody technology. The Rabbitts group has developed methodology for screening for functional intrabodies by yeast two hybrid methodology (Tanaka, T., et al., J Mol Biol 2003 331, 1109-1120; Tanaka, T. & Rabbitts, T. H. Embo J 2003 22, 1025-1035; Tanaka, T., et al., Nucleic Acids Res 2003 31, e23; Tse, E. et al. J Mol Biol 2002 317, 85-94; Rabbitts, T. H. et al. Blood Cells Mol Dis 2001 27, 249-259). In recognition of the difficulty of identifying antibodies that function in the reducing cytoplasmic environment, these investigators advocate screening for and testing antibodies directly in this environment. Although these investigators have noted the superior expression properties for single domain intrabodies (Tanaka, T., et al., J Mol Biol 2003 331, 1109-1120), the yeast two hybrid is a qualitative method in general, and has not been applied to quantitative affinity screening.

Barberis et al. have developed frameworks that are stable in intracellular expression, and then have used yeast two hybrid-based screens to identify functional intrabodies (U.S. patent application 20010024831 and also Auf der Maur, A., et al., Methods 2004 34, 215-224; der Maur, A. A. et al. J Biol Chem 2002 277, 45075-45085; Auf der Maur, A., et al., FEBS Lett 2001 508, 407-412).

Plückthun et al., have engineered single-chain antibodies for improved expression in the absence of disulfide bonds by directed evolution and phage display (Proba, K., et al., J Mol Biol 1998 275, 245-253. Although each of these strategies has helped advance intrabody technology, each fails to allow production of stable intrabodies that provide a high enough binding affinity for efficient and successful use.

Numerous diseases and disorders, including neurological disorders and cancers are recognized as potential targets for intrabody-based therapeutic methods, but presently there is a lack of suitable intrabodies available for use in such therapeutic methods.

One neurological disease that has received much study is Huntingon's disease. Cellular and genetic characteristics of huntingtin polypeptide aggregation-associated disorders have begun to be elucidated. It is known that Huntington's disease is characterized by mutant huntingtin protein with abnormal expanses of polyglutamine tracts. The normal, wild-type huntingtin protein has uninterrupted tracts of glutamine residues encoded by CAG triplet repeats. It now known that the expansion of the length of these uninterrupted tracts or regions of glutamine repeats in proteins is associated with specific neurodegenerative diseases, such as Huntington's disease. The expansion of polyglutamine tracts in proteins may become pathogenic if the polyglutamine tracts expand beyond a threshold length, which for most disorders associated with polyglutamine expansion is a length of approximately 35-40 residues. When the expansion threshold is reached, the presence of the abnormal protein is associated with neurodegenerative diseases such as: Huntington's disease. In this disorder, abnormal expanded regions of CAG repeats have been identified in the coding region of a protein.

In addition to the expanded repeats, the N-terminal region of the huntingtin gene product, huntingtin (Htt), forms beta-sheet rich aggregates in striatal neurons. The Htt aggregation correlates with the number of CAG repeats as does the patient's age of onset of Huntington's disease. Features of the Htt aggregates include the co-aggregation of the Htt fragments with transcription factors, such as CBP (Nucifora, F. C., et al., Science Mar. 23 2001;291(5512):2423-8) as well as the interference with the cellular protein degradation system by the Htt aggregates (Bence, N. F., et al, Science 2001 May 25;292(5521):1552-5).

Features of Huntington's disease include the gradual loss of neurons with a concomitant loss of motor and cognitive functions. In addition, the onset of Huntington's disease is characterized by choreic movements that result from the selective involvement of medium spiny neurons of the striatum. As Huntington's disease progresses, more regions of the brain and spinal cord of the patient become involved. The severity of the symptoms and progression of huntingtin polypeptide aggregation-associated diseases varies from patient to patient, in part due to fact that the length of the expanded polyglutamine region correlates with the severity of the symptomatic presentation. Thus, patients with longer expanded polyglutamine regions may have more severe clinical effects from the disease and may show an earlier age of onset than would patients with shorter expanded polyglutamine regions. Huntington's normally presents symptomatically in mid to late life and is dominantly inherited.

Huntington's disease is neurodegenerative and fatal, and although it is possible to diagnose Huntington's disease, there are very limited treatment options available for patients diagnosed with the disorder. The lack of effective treatments for Huntington's disease means that even with a definitive diagnosis, the therapeutic options are quite limited. A number of antibodies that specifically bind to regions of the huntingtin protein have been identified but various features of the antibodies limit their effectiveness for the treatment of Huntington's disease. Such antibodies are generally not stable in the reducing environment of the cytoplasm of cells, which results in poor expression and low antibody activity even if expressed intracellularly. Similarly, antibody-related therapeutics for other diseases also are limited by antibody characteristics that limit expression, stability, and/or affinity in the intracellular environment. Therefore, there a need exists for more effective antibodies for use in the treatment of Huntington's disease and in many other diseases associated with abnormal protein activity.

### SUMMARY OF THE INVENTION

The invention provides novel antibodies (also referred to herein as intrabodies) as well as newly discovered methods and products relating to the production and use of intrabodies. The methods of the invention for producing effective intrabodies differ from those in the prior art. For example, the methods of the invention differ markedly from those proposed by Rabbitts et al., because we have chosen to mimic an important biophysical parameter (reducing redox potential) by genetically removing the disulfide bond. We then directly select for improved affinity in a system (yeast surface display), which in contrast to the Rabbitts system, is better suited to such quantitative screens (Boder, E. T. & Wittrup, K. D Nat Biotechnol 1997 15, 553-557; Boder, E. T. & Wittrup, K. D. Biotechnol Prog 1998 14, 55-62; Boder, E. T., et al., Proc Natl Acad Sci USA 2000 97, 10701-10705; Boder, E. T. & Wittrup, K. D. Methods Enzymol 2000 328, 430-444; Colby, D. W. et al. Methods Enzymol 2004 388, 348-358; Colby, D. W. et al. J Mol Biol 2004 342, 901-912; VanAntwerp, J. J. & Wittrup, K. D. Biotechnol Prog 2000 16, 31-37).

Similarly, rather than identifying a single scaffold and randomizing CDR loops as done by Barberis et al., our methods include making any given antibody domain to mimic its binding phenotype in the intracellular environment by mutation of cysteine residues, followed by affinity maturation of the non-disulfide-containing domain. Thus, the methods of the invention include the genetic removal of one or more disulfide bond(s) and then selection of functional antibodies by directed evolution methods. Unlike the methods proposed by Pluckthun et al., which simply sought functional expression, not improved affinity, we have also discovered methods that relate to improving affinity under reducing conditions by affinity maturing an antibody in which the disulfide has been genetically deleted.

One of our most surprising discoveries is that elimination of a disulfide bond in a domain antibody (dAb) can decrease binding affinity by over one thousand fold without substantially altering expression. Previously, it had been generally thought that the primary importance of the disulfide bond in antibody variable domains was with respect to stability, not binding (Glockshuber, R., et al., Biochemistry 1992 31, 1270-1279). Although stability is necessary to create a functional intrabody, it is not sufficient to improve affinity; one can stabilize an antibody variable domain without altering or improving its affinity (see, Graff et al., Prot. Eng. Des. Sel. 2004 17(3):293-304). For example, Jermutus & Pluckthun described tailoring ribosome display selections towards stability by the use of reducing agents, or towards binding affinity by off-rate selection (Jermutus, L., et al., Proc Natl Acad Sci U S A 2001 98, 75-80. The importance of stability to intrabody function has been emphasized in the past (Worn, A. et al. J Biol Chem 2000 275, 2795-2803). In contrast; our discovery indicates that loss of binding affinity in a disulfide-free but stable intrabody can be a dominant problem to overcome for functional intrabodies, and the novel methods and products of the invention enable production of functional, high-affinity single-domain disulfide-independent antibodies.

We have identified antibodies and antigen-binding fragments thereof (also referred to herein as intrabodies) that specifically bind to a target protein in vitro and in vivo and have features that increase their affinity, expression, and stability. These features result in improved usefulness of antibodies and antigen-binding fragments thereof in therapeutic and diagnostic applications. We have surprisingly discovered that antibodies in which disulfide bonds are removed, such as single chain (e.g. scFv) and single domain (e.g. VL) antibodies, can retain or surpass the expression, stability, and affinity of the [parent] antibody in the reducing environment inside cells. For illustrative purposes, the description provided herein relates in part to antibodies we have discovered that specifically bind huntingtin protein (Htt) and are useful to detect Htt and/or are useful to modulate Htt activity. These antibodies are useful in the methods of the invention relating to the treatment and/or prevention of Huntington's disease (HD). Other antibodies that bind to other proteins also are enhanced by the invention.

The methods of the invention are also useful to make and use antibodies that specifically bind to target proteins other than Htt. Thus, the methods of the invention can be used to identify and use antibodies, for example, disulfide-independent antibodies, to target proteins that are associated with disease states other than HD. The methods disclosed herein are intended to relate to antibodies or antigen-binding fragments thereof that can be used to modulate and/or label proteins associated with the onset, progression, or regression of diseases and disorders in addition to HD.

We have identified single domain antibodies that are useful to inhibit Htt polypeptide aggregation. Our findings show that the identified antibodies can be used to inhibit aggregation of Htt polypeptide. In addition, the identified antibodies are useful for localization of Htt in vivo and in vitro. Some of the identified antibodies are also useful for assessing candidate agents for their ability to reduce Htt aggregation. We have discovered that a immunoglobulin light chain polypeptide sequence, for example one that includes SEQ ID NO:1, by itself can bind to Htt protein and can also function to inhibit Htt aggregation. We have also removed disulfide bonds from the antibody and modified the antibody sequence and thereby greatly increased the affinity of the disulfide-free antibody to about 10 nM or less.

Advantages of the disulfide-independent single domain antibodies or antigen-binding fragments of the invention are that they are expressed at a high level in cells, are stable in an intracellular environment, and have a high affinity. Accordingly, the invention provides in certain aspects novel methods of treating diseases, (e.g. HD) by using the antibodies of the invention to modulate their target proteins. In the case of HD, antibodies of the invention inhibit Htt aggregation. The novel antibodies of the invention are also useful for mapping the localization of their target protein in vivo and/or in vitro and for target validation. For example, Htt-binding antibodies or antigen-binding fragments of the invention can be used to detect the presence and location of their target molecule, Htt. The antibodies or antigen-binding fragments thereof of the invention can be delivered to cells and/or subjects using a number of techniques. These techniques include the use of an expression vector that encodes an antibody or antigen-binding fragment of the invention to express the antibody intracellularly. The antibodies or antigen-binding fragments thereof of the invention can also be administered through the use of peptide transduction domains to deliver an antibody of the invention into cells.

In addition to the use of the antibodies of the invention for treatment of HD, we have also determined that the removal of the disulfide bonds in other single domain antibodies may be used in methods and compositions useful for the prevention and/or treatment of other disorders, including, but not limited to neurological disorders, HIV, and cancer.

According to one aspect of the invention, isolated single domain antibodies or antigen-binding fragments thereof are provided that are disulfide-independent antibodies or disulfide-independent antigen-binding fragments thereof. In some embodiments, the single-domain antibodies or antigen-binding fragments thereof are disulfide-free antibodies or disulfide-free antigen-binding fragments thereof. In some embodiments, the antibody affinity is between about 50 nM and about 5 nM. In certain embodiments, the antibody affinity is at least about 10 nM. In some embodiments, wherein the antibody or antigen-binding fragment thereof is linked to a targeting molecule. In some embodiments, the targeting polypeptide is a nuclear localization sequence (NLS). In some embodiments, the targeting molecule's target is a neuronal cell. In certain embodiments, the antibody or antigen-binding fragment thereof is linked to a protein transduction domain (PTD). In some embodiments, the PTD is selected from the group consisting of: a TAT protein, antennepedia protein, and synthetic poly-arginine. In some embodiments, the antibody or antigen-binding fragment thereof is linked to a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP). In certain embodiments, the antibody or antigen-binding fragment thereof comprises a single light chain polypeptide comprising the amino acid sequence set forth as SEQ ID NO: 1 or SEQ ID NO:2. In some embodiments, the antibody or antigen-binding fragment thereof comprises a single light chain polypeptide comprising the amino acid sequence set forth as SEQ ID NO:10. In some embodiments, the antibody or antigen-binding fragment thereof comprises an amino acid sequence that is a fragment of the amino acid sequence set forth as SEQ ID NO: 3 or SEQ ID NO:4. In some embodiments, the antibody or antigen-binding fragment thereof inhibits huntingtin aggregation. In some embodiments, the antibody or antigen-binding fragment thereof specifically binds the N-terminus of huntingtin protein. In certain embodiments, the antibody specifically binds the region of the N-terminus encoded by exon 1 of the huntingtin gene.

According to another aspect of the invention, isolated antibodies or antigen-binding fragments thereof that specifically binds to an epitope on huntingtin protein are provided. The antibodies or antigen-binding fragments thereof include a single light chain polypeptide comprising the amino acid sequence set forth as SEQ ID NO: 1, SEQ ID NO:2, or SEQ ID NO:10. In some embodiments, a variant of one of the foregoing isolated antibody or antigen-binding fragment thereof is provided. In some embodiments, from about one to ten amino acids of the variant differ from the amino acids in the sequences set forth as SEQ ID NO:1, SEQ ID NO:2, or SEQ ID NO:10. In some embodiments, the antibody or antigen-binding fragment thereof includes an amino acid sequence that is a fragment of the amino acid sequence set forth as SEQ ID NO: 3 or SEQ ID NO:4. In some embodiments, a variant of an aforementioned isolated antibody or antigen-binding fragment thereof is provided. In some embodiments, from about one to ten amino acids of the variant differ from the amino acids in a sequence set forth as SEQ ID NO:3 or SEQ ID NO:4. In some embodiments, the antibody or antigen-binding fragment thereof is a disulfide-independent antibody. In some embodiments, the disulfide-independent antibody or antigen-binding fragment thereof is a disulfide-free antibody or antigen-binding fragment thereof. In certain embodiments, the antibody or antigen-binding fragment thereof affinity is at least about 10 nM. In some embodiments, the antibody or antigen-binding fragment thereof inhibits huntingtin aggregation. In certain embodiments, the antibody or antigen-binding fragment thereof specifically binds the N-terminus of huntingtin protein. In some embodiments, the antibody or antigen-binding fragment thereof specifically binds the region of the N-terminus encoded by exon 1 of the huntingtin gene. In some embodiments, the antibody or antigen-binding fragment thereof is linked to a targeting molecule. In some embodiments, the targeting molecule's target is a neuronal cell. In some embodiments, the antibody or antigen-binding fragment is linked to a protein transduction domain (PTD). In certain embodiments, the PTD is selected from the group consisting of: a TAT protein, antennepedia protein, and synthetic poly-arginine. In some embodiments, the antibody or antigen-binding fragment is linked to a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP).

The invention also provides, in some aspects, expression vectors that include a nucleotide sequence encoding any of the foregoing antibodies or antigen-binding fragments thereof are provided. In some embodiments, the vector is an adenovirus vector. In certain embodiments, the adenovirus vector is pACCMV2. In some embodiments, the expression vector also includes a nucleotide sequence encoding a targeting polypeptide. In some embodiments, the targeting polypeptide is a nuclear localization sequence (NLS). In certain embodiments, the expression vectors also include a nucleotide sequence encoding a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP). In some embodiments, an isolated host cell transformed or transfected with any of the foregoing expression vectors is provided.

According to yet another aspect of the invention, isolated nucleic acid molecule that include a nucleotide sequence that encodes SEQ ID NO:1, SEQ ID NO:2, or SEQ ID NO:10 are provided.

According to another aspect of the invention, isolated nucleic acid molecules that include a nucleotide sequence that encodes SEQ ID NO:3 or SEQ ID NO:4 are provided.

According to another aspect of the invention, methods of making a disulfide-independent single-domain antibody are provided. The methods include obtaining a single-domain antibody that specifically binds to a target protein, mutating at least one or more cysteine amino acids in the antibody, wherein the cysteine mutation removes one or more disulfide bonds from the antibody, applying directed evolution to the amino acid sequence of the antibody, and contacting the directly evolved antibody with the target protein to determine specific binding of the directly evolved antibody to the target protein, wherein the directly evolved antibody is a disulfide-free single domain antibody. In some embodiments, the disulfide-independent antibody or antigen-binding fragment thereof is a disulfide-free antibody or antigen-binding fragment thereof. In some embodiments, the directed evolution comprises yeast surface display. In certain embodiments, the directed evolution comprises phage display. In some embodiments, the directed evolution comprises ribosome display. In certain embodiments, the directed evolution comprises error-prone PCR. In some embodiments, the directed evolution comprises nucleotide analogue PCR. In some embodiments, the directed evolution comprises DNA shuffling. In some embodiments, the directed evolution increases the affinity of the disulfide-independent single-domain antibody for the target protein. In certain embodiments, the method of making a disulfide-independent, single-domain antibody also includes applying directed evolution one or more additional times. In some embodiments, the disulfide-independent single domain antibody has an affinity that is between about 50 nM and about 5 nM. In certain embodiments, the disulfide-independent single domain antibody has an affinity that is at least about 10 nM. In some embodiments, the method of making a disulfide-independent, single-domain antibody also includes linking the disulfide-independent single domain antibody to a targeting molecule. In some embodiments, the targeting polypeptide is a nuclear localization sequence (NLS). In some embodiments, the targeting molecule's target is a neuronal cell. In some embodiments, the method of making a disulfide-independent, single-domain antibody also includes linking the disulfide-independent single domain antibody to a protein transduction domain (PTD). In some embodiments, the PTD is selected from the group consisting of: a TAT protein, antennepedia protein, and synthetic poly-arginine. In some embodiments, making the disulfide-independent, single-domain antibody also includes linking the disulfide-independent single domain antibody to a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP). In certain embodiments, the disulfide-independent single domain antibody inhibits huntingtin aggregation. In some embodiments, the disulfide-independent single domain antibody specifically binds the N-terminus of huntingtin protein. In some embodiments, the disulfide-independent single domain antibody specifically binds the region of the N-terminus encoded by exon 1 of the huntingtin gene. In certain embodiments, the disulfide-independent single-domain antibody is any of the foregoing single-domain antibodies or antigen-binding fragments thereof provided.

According to another aspect of the invention, expression vectors that include a nucleotide sequence encoding the disulfide-independent, single-domain directly evolved antibody of any of the foregoing claims are provided. In some embodiments, the vector is an adenovirus vector. In certain embodiments, the adenovirus vector is pACCMV2. In some embodiments, the disulfide-independent single-domain antibody or antigen-binding fragment thereof is a single-domain antibody or antigen-binding fragment thereof of any of the foregoing aspects of the invention. In some embodiments, the expression vector also includes a nucleotide sequence encoding a targeting polypeptide. In some embodiments, the targeting polypeptide is a nuclear localization sequence (NLS). The invention also provides in some aspects, isolated host cells transformed or transfected with any of foregoing expression vectors.

According to yet another aspect of the invention, methods of preventing or treating a disease in a subject are provided. The methods include administering any of the foregoing antibodies or antigen-binding fragments thereof, or any antibody or antigen-binding fragment thereof made by any of the foregoing methods, or any of the foregoing expression vectors to a subject in need of such treatment in an amount effective to prevent or treat the disease in the subject. In some embodiments, the disease is a neurological disease. In certain embodiments, the neurological disease is selected from the group that consists of Huntington's disease, Alzheimer's disease, and Parkinson's disease. In certain embodiments, the disease is HIV or cancer. In some embodiments, the mode of administration is selected from the group consisting of: implantation, mucosal administration, intramuscular injection, intravenous injection, subcutaneous injection, intrathecal administration, inhalation, and oral administration. In certain embodiments, the antibody or antigen-binding fragment thereof is administered in combination with an additional drug or therapy for the disease. In some embodiments, the subject is a human. In some embodiments, the subject has been diagnosed with the disease or is at risk of developing the disease. In certain embodiments, the antibody or antigen-binding fragment thereof is linked to a targeting molecule. In some embodiments, the targeting molecule comprises a protein transduction domain (PTD). In some embodiments, the targeting molecule's target is a neuronal cell. In some embodiments, the antibody or antigen-binding fragment thereof is labeled with one or more cytotoxic agents.

According to yet another aspect of the invention, methods of preventing or treating a disease in a subject are provided. The methods include administering any of the foregoing expression vectors of the invention, or a nucleic acid that encodes any of the foregoing antibodies or antigen-binding fragments thereof of the invention, or encodes an antibody or antigen-binding fragment thereof made by any of the foregoing methods of the invention that specifically binds to the protein, in an amount effective to modulate the activity of the protein. In some embodiments the nucleic acid is in an expression vector. In some embodiments, the expression vector is an adenovirus vector. In some embodiments, the adenovirus vector is pACCMV2. In some embodiments, the expression vector further comprises a nucleotide sequence encoding a targeting polypeptide. In some embodiments, the targeting polypeptide is a nuclear localization sequence (NLS). In some embodiments, the expression vector further comprises a nucleotide sequence encoding a reporter polypeptide. In certain embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP).

According to yet another aspect of the invention, methods of modulating activity of a protein are provided. The methods include contacting a cell intracellularly with any of the foregoing expression vectors, or any of the foregoing antibodies or antigen-binding fragments thereof, or an antibody or antigen-binding fragment thereof made by any of the foregoing methods, that specifically binds to the protein in an amount effective to modulate the activity of the protein. In some embodiments, the protein is in a cell. In certain embodiments, modulating is inhibiting activity of the protein. In some embodiments, modulating is enhancing activity of the protein. In some embodiments, the cell is a neuronal cell. In some embodiments, the cell is intracellularly contacted with an antibody or antigen-binding fragment thereof linked to a targeting molecule. In some embodiments, the targeting molecule is a protein transduction domain (PTD). In some embodiments, the PTD is selected from the group consisting of: a TAT protein, antennepedia protein, and synthetic poly-arginine. In certain embodiments, the targeting molecule's target is a neuronal cell. In some embodiments, the antibody or antigen-binding fragment thereof is linked to a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP). In some embodiments, the antibody or antigen-binding fragment thereof is labeled with one or more cytotoxic agents.

According to yet another aspect of the invention, methods of modulating activity of a protein are provided. The methods include contacting a cell intracellularly with a nucleic acid that encodes any of the foregoing antibodies or antigen-binding fragments thereof, or an antibody or antigen-binding fragment thereof made by the any of the foregoing methods, that specifically binds to the protein, in an amount effective to modulate the activity of the protein. In some embodiments, the nucleic acid is in an expression vector. In certain embodiments, the vector is an adenovirus vector. In some embodiments, the adenovirus vector is pACCMV2. In certain embodiments, the expression vector further comprises a nucleotide sequence encoding a targeting polypeptide. In some embodiments, the targeting polypeptide is a nuclear localization sequence (NLS). In some embodiments, the expression vector also includes a nucleotide sequence encoding a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP).

According to yet another aspect of the invention, methods of treating or preventing Huntington's Disease (HD) in a subject are provided. The methods include administering any of the foregoing antibodies or antigen-binding fragments thereof or any of the foregoing expression vectors to a subject in need of such treatment in an amount effective to inhibit huntingtin aggregation in the subject. In some embodiments, the mode of administration is selected from the group consisting of: implantation, mucosal administration, intramuscular injection, intravenous injection, subcutaneous injection, intrathecal administration, inhalation, and oral administration. In certain embodiments, the antibody or antigen-binding fragment is administered in combination with an additional drug or therapy for treating Huntington's disease. In some embodiments, the subject is a human. In some embodiments, the subject has been diagnosed with Huntington's disease or is at risk of developing Huntington's disease. In some embodiments, the antibody or antigen-binding fragment is linked to a targeting molecule. In certain embodiments, the targeting molecule comprises a protein transduction domain (PTD). In some embodiments, the targeting molecule's target is a neuronal cell. In some embodiments, the antibody or antigen-binding fragment is labeled with one or more cytotoxic agents.

According to another aspect of the invention, methods of inhibiting aggregation of huntingtin protein in a cell are provided. The methods include contacting the cell intracellularly with any of the foregoing antibodies or antigen-binding fragments or any of the foregoing expression vectors in an amount effective to inhibit huntingtin aggregation in the cell. In some embodiments, the cell is a neuronal cell. In certain embodiments, the cell is intracellularly contacted with an antibody or antigen-binding fragment linked to a targeting molecule. In some embodiments, the targeting molecule is a protein transduction domain (PTD). In some embodiments, the PTD is selected from the group consisting of: a TAT protein, antennepedia protein, and synthetic poly-arginine. In certain embodiments, the targeting molecule's target is a neuronal cell. In some embodiments, the antibody or antigen-binding fragment is linked to a reporter polypeptide. In some embodiments, the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP). In certain embodiments, the antibody or antigen-binding fragment is labeled with one or more cytotoxic agents.

According to yet another aspect of the invention, methods of diagnosing a disease or disorder in a subject are provided. The methods include contacting a sample obtained from a subject with any of the foregoing disulfide-independent, single-domain antibodies or antigen-binding fragments thereof or any of the foregoing method of the invention, determining a level of the protein to which the disulfide-independent, single-domain antibody or antigen binding fragment thereof specifically binds, comparing the level obtained to a control level, wherein a difference in the level obtained and the control level is diagnostic for the disease or disorder in the subject. In some embodiments, the disulfide-independent antibody or antigen-binding fragment thereof is a disulfide-free antibody or antigen-binding fragment thereof.

According to yet another aspect of the invention, methods of evaluating a treatment for a disease or disorder in a subject are provided. The methods include contacting a first sample obtained from a subject with any of the foregoing disulfide-independent, single-domain antibodies or antigen-binding fragments thereof or made with any of the foregoing methods, determining a level of the protein to which the disulfide-independent, single-domain antibody or antigen binding fragment thereof specifically binds, contacting a second sample obtained from the subject at least one day after the first sample with the disulfide-independent, single-domain antibody or antigen-binding fragment thereof, determining the level of the protein to which the disulfide-independent, single-domain antibody or antigen binding fragment thereof specifically binds, comparing the first sample level to the second sample level, wherein a difference in the first sample level and the second sample level is an evaluation of the treatment. In some embodiments, the disulfide-independent antibody or antigen-binding fragment thereof is a disulfide-free antibody or antigen-binding fragment thereof. In certain embodiments, the method also includes selecting a treatment for the disease or disorder based on the evaluation of the disease.

According to yet another aspect of the invention, methods for determining onset, progression, or regression of a disease or disorder are provided. The methods include contacting a sample obtained from a subject with any of the foregoing disulfide-independent, single-domain antibodies or antigen-binding fragments thereof or made with any of the foregoing methods, determining a level of the protein to which the disulfide-independent, single-domain antibody or antigen binding fragment thereof specifically binds, comparing the level obtained to a control level, wherein a difference in the level obtained and the control level a determination of onset, progression, or regression of the disease or disorder in the subject. In some embodiments, the disulfide-independent antibody or antigen-binding fragment thereof is a disulfide-free antibody or antigen-binding fragment thereof.

According to yet another aspect of the invention, methods of diagnosing a disease or disorder in a subject are provided. The methods include administering to a subject any of the foregoing disulfide-independent, single-domain antibodies or antigen-binding fragments thereof or made with any of the foregoing methods, determining a level of the protein to which the disulfide-independent, single-domain antibody or antigen binding fragment thereof specifically binds in the subject, comparing the level obtained to a control level, wherein a difference in the level obtained and the control level is diagnostic for the disease or disorder in the subject. In some embodiments, the disulfide-independent antibody or antigen-binding fragment thereof is a disulfide-free antibody or antigen-binding fragment thereof.

According to yet another aspect of the invention, methods of evaluating a treatment for a disease or disorder in a subject are provided. The methods include administering a first time to a subject any of the foregoing disulfide-independent, single-domain antibodies or antigen-binding fragments thereof or made with any of the foregoing methods, determining a first level of the protein to which the disulfide-independent, single-domain antibody or antigen binding fragment thereof specifically binds in the subject, administering a second time to the subject a disulfide-independent, single-domain antibody or antigen-binding fragment thereof, wherein the subsequent time is at least one day after the first time, determining the second level of the protein to which the disulfide-independent, single-domain antibody or antigen binding fragment thereof specifically binds in the subject, comparing the first level to the second level, wherein a difference in the first level and the second level obtained is an evaluation of the treatment. In some embodiments, the method also includes selecting a treatment for the disease or disorder based on the evaluation of the disease. In some embodiments, the disulfide-independent antibody or antigen-binding fragment thereof is a disulfide-free antibody or antigen-binding fragment thereof.

According to another aspect of the invention, methods for determining onset, progression, or regression of a disease or disorder are provided. The methods include contacting a sample obtained from a subject with any of the foregoing disulfide-independent, single-domain antibodies or antigen-binding fragments thereof or made with any of the foregoing methods, determining a level of the protein to which the disulfide-independent, single-domain antibody or antigen binding fragment thereof specifically binds, comparing the level obtained to a control level, wherein a difference in the level obtained and the control level a determination of onset, progression, or regression of the disease or disorder in the subject. In some embodiments, the disulfide-independent antibody or antigen-binding fragment thereof is a disulfide-free antibody or antigen-binding fragment thereof.

The use of the foregoing antibodies and antigen-binding fragments thereof in the preparation of a medicament, particularly a medicament for prevention and/or treatment of a protein-associated disorder, including but not limited to Huntington's disease, Alzheimer's disease, Parkinson's disease, neurological diseases/disorders, protein-aggregation disorders, HIV, or cancer is also provided.

These and other objects of the invention will be described in further detail in connection with the detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction an the arrangement of components set forth in the following description or illustrated in the drawings. The invention is capable of other embodiments and of being practiced or o being carried out in various ways.

### BRIEF DESCRIPTION OF THE DRAWINGS

The figures are illustrative only and are not required for enablement of the invention disclosed herein.

FIG. 1 is a histogram of results indicating that the single domain antibody inhibits htt aggregation in stoichiometric fashion.

FIG. 2 shows affinity graphs of the FL1 channel, which measures expression of the antibody on the yeast cell surface, and the FL2 channel, which reflects binding to the huntingtin peptide antigen. FIG. 2A shows the affinity with disulfide bonds and FIG. 2B shows the affinity without the disulfide bonds. Knocking out the disulfide bond reduced the affinity from 30-50 nM to the micromolar range, indicating that binding under the reducing conditions of the cell would be weak.

FIG. 3 is graph of results of flow cytometry measurements of yellow fluorescent protein (YFP) fluorescence measured 24 hours post induction. Compared to the expression of ScFv-YFP, the single domain intrabody (SDIb-YFP) was expressed at much higher levels in the cytoplasm, as measured by YFP fluorescence observed using antibody-YFP fusion proteins in yeast.

FIG. 4 is an affinity plot of SDIb labeled at 1 nM huntingtin peptide. The FL1 channel measures expression of the antibody on the yeast cell surface, the FL2 channel reflects binding to the huntingtin peptide antigen.

FIG. 5 is a histogram of data obtained in a study to compare aggregation of huntingtin protein in the presence and absence of the engineered single-domain intrabody. Aggregation inhibition resulting from co-expression of engineered intrabody with Htt-x1-Q97-YFP.

FIG. 6 is a schematic drawing of yeast surface display. The single chain antibody is expressed as a fusion to the Aga2 mating protein. C-myc and HA epitope tags are present to quantify expression by immunofluorescence.

FIG. 7 are schematic drawings of the plasmid map of pCTCON. FIG. 7A is a diagram of CON cd20, which is expressed from the plasmid as a fusion to the yeast mating protein Aga2. FIG. 7B illustrates the position at which the CON cd20 gene can be replaced with an scFv of interest using the NheI and BamHI sites.

FIG. 8 is a drawing of a sort gate. If a diagonal population was present in the library, a sort gate such as the one labeled R7 was drawn to take full advantage of expression normalization.

FIG. 9 is a graph of an antibody display for GST-GFP, an antibody specific for exon I of Htt. The antibodies is an anti-huntingtin scFv isolated from the library.

FIG. 10 is a graph of an antibody display for GST-HttQ67-GFP, an antibody specific for exon I of Htt. The antibody is an anti-huntingtin scFv isolated from the library.

FIG. 11 shows a schematic yeast HD FRET model.

FIG. 12 shows yeast HD FRET model constructs.

FIG. 13 shows a schematic diagram of an expression vector into which antibodies were subcloned.

FIG. 14 shows graphs of the fluorescent spectrophotometry results of FRET using anti-htt antibodies.

FIG. 15 shows a formula and schematic diagrams used to (FIG. 15A) determine aggregation inhibition properties and (FIG. 15B) to use directed evolution techniques to optimize antibodies.

FIG. 16 shows a histogram that illustrates the results of affinity maturation of antibodies GST-HttQ67-GFP.

FIG. 17 is a graph illustrating binding curves that show that the antibody affinity improved over 5000-fold after two rounds of mutagenesis and screening.

FIG. 18 shows a list and diagram showing that the best clone acquired mutations through both DNA shuffling and error-prone PCR.

FIG. 19 is a graph of the binding activity of the V_{L} domain of 2.4.3.

FIG. 20 shows schematic diagrams and graphs indicating that the single-domain antibody was well expressed in cytoplasm as YFP fusion FIG. 21 shows logarithmic graphs of binding of sulfide-containing antibody to disulfide-free antibody. The disulfide knock-outs bind less strongly.

FIG. 22 is a graph indicating the binding affinity of the affinity matured disulfide-free antibody.

FIG. 23 shows a graph that demonstrates the binding and aggregation ability of three engineered anti-Htt antibodies.

FIG. 24 shows histograms and a graph depicting results obtained from a single domain intrabody against huntingtin was engineered for high affinity in the absence of a disulfide bond. FIG. 24A shows histograms of yeast cell surface expression levels for V_{L} and V_{L} C22V C89A, indicating comparable levels of expression with and without the disulfide bond. FIG. 24B is a graph showing antigen binding curves for yeast surface displayed V_{L} mutants measured by flow cytometry. Values normalized to maximal intensity measured, except for V_{L},C22V, C89A, which was normalized to maximal intensity measured for V_{L}. V_{L} (diamonds) has a Kd of approximately 30 nM, while V_{L} with cysteine mutations (V_{L},C22V, C89A, circles) has significantly lower binding affinity (>10 mM). Repeated rounds of random mutagenesis of V_{L},C22V, C89A followed by sorting for improved binding resulted in the mutant V_{L}12.3, which has a Kd of approximately 3 nM. FIG. 24C is a histogram that shows the effect of V_{L} and V_{L},C22V, C89A on htt aggregation in ST14A cells transiently transfected with indicated intrabody or vector control and httex1Q97-GFP at a 2:1 plasmid ratio. Both intrabodies are equally capable of partially blocking aggregation when overexpressed at high levels. *** p<0.001. FIG. 24D is a schematic homology model that includes mutations obtained during engineering; model contains residues present before mutagenesis. Mutations observed after mutagenesis and sorting were F371, Y51D, K67R, and A75T.

FIG. 25 shows results indicating that engineered V_{L}12.3 robustly blocks htt aggregation in several different cell lines. FIG. 25A. shows a graph of counts of visible aggregates in ST14A cells transiently co-transfected with httex1Q97-GFP and either an intrabody (C4 (3) or V_{L}12.3) or an empty control vector. Cells with visible aggregates were counted 1, 2 and 3 days post-transfection (5:1 intrabody:htt plasmid ratio, N=3). V_{L}12.3 (circles) persistently eliminated htt aggregation over three days. FIG. 25B shows a graph of the dose response of V_{L}12.3 that was measured at two days by varying intrabody:htt plasmid ratios (N=3). FIG. 25C is a digitized image of fluorescence microscopy images of typical cells. FIG. 25D shows flow cytometry histograms that show expression level per cell of httex1Q97-GFP in transfected cells in the presence of intrabody compared to empty vector (mean fluorescence intensity 82 MFU vs. 76 MFU, respectively; transfection efficiencies were comparable in both samples, at 13% and 11%, respectively). FIG. 25E is a histogram comparison of intrabody activity for the intrabodies mentioned above and a non-huntingtin binding intrabody (scFv ML3.9) and wild-type V_{L}, at 1:1 intrabody:htt plasmid ratio (***, p<0.001) in SH-SY5Y human neuroblastoma cells. FIG. 25F is a histogram showing a partial dose response for the same intrabodies in HEK293 cells. FIG. 25G shows a digitized image of a western blot of Triton-soluble and Triton-insoluble fractions of cells lysed 24 hours after cotransfection at a 2:1 intrabody:htt ratio. FIG. 25H shows a digitized image of an anti-His6 western blot of intrabody expression levels in transiently transfected HEK293 cells.

FIG. 26 shows results of FACs analysis and a histogram indicating that engineered intrabody V_{L}12.3 inhibits metabolic dysfunction in neuronal model of HD. ST14A cells were transfected with a plasmid encoding either GFP, httex1Q25-GFP, httex1Q97-GFP, or httex1Q97-GFP with V_{L}12.3 in a 2:1 ratio. FIG. 26A shows results of live GFP-positive cells were collected by FACS in a 96-well plate, 30,000 cells per well 48 hrs posttransfection; typical dot plot is shown for a GFP sample. Other samples showed similar pattern, and the sorting gate (box shown) was the same in all instances. FIG. 26B is a histogram of results from cells incubated with MTT reagent for 3 hours, solubilized, and the A570 was measured. Mean values from 3 separate experiments containing all four samples are shown. Statistics directly over error bars are for comparison to GFP, ns, not significant, * p<0.05, ** p<0.01. Statistics over brackets are comparisons between the two samples indicated. Four additional pairwise comparisons may be made between httex1Q97-GFP and httex1Q97-GFP+V_{L}12.3; the pooled results indicate a 56±25% increase in A570, p<0.001. Expression of httex1Q97-GFP significantly reduced the ability of cells to reduce MTT, but this effect was reversed by the co-expression of V_{L}12.3.

FIG. 27 shows results indicating that V_{L}12.3 suppresses aggregation and rescues toxicity in a S. cerevisiae model of HD. FIG. 27A shows a digitized image of a filter retardation assay showing httex1Q72-CFP aggregates (dark) from lysates of cells expressing httex1Q25-CFP or httex1Q72-CFP with either V_{L}12.3 or an empty vector control. Dashed circles indicate where insoluble material would appear. Difference between 25Q with and without V_{L}12.3 is insignificant and within variance usually observed for the assay. FIG. 27B shows spottings of yeast strains indicating ability to grow on solid media. FIG. 27C is a graph showing growth curves obtained by measuring the optical density of yeast cultures at 600 nm. Yeast expressing V_{L}12.3-YFP along with httex1Q72-CFP grow at rates comparable to those expressing htt with non-pathological polyglutamine repeat lengths, in contrast to those carrying an empty vector only.

FIG. 28 is a histogram showing that AD-V_{L}12.3 reduces huntintinQ103-GFP aggregation in a neuronal cell culture model of Huntington's disease.

### DESCRIPTION OF THE SEQUENCES

SEQ ID NO:1 is a single domain antibody engineered for intracellular expression and high affinity:

QPVLTQSPSVSAAPRQRVTISCSGSNSNIGSNTVNWFQQLPGRAPELLMY
YDDLLAPGVSDRFSGSKSGTSASLAISGLQSEDEADYYCATWDDSLNGWV
FGGGTKVTVLS.

SEQ ID NO:2 is a single domain antibody without a disulfide bond that was engineered for intracellular expression and high affinity:

SRPVLTQSPSVSAAPRQRVTISVSGSNSNIGSNTVNWIQQLPGRAPELLM
YDDDLLAPGVSDRFSGSRSGTSASLTISGLQSEDEADYYAATWDDSLNDWV
FGGGTKVTVLS.

SEQ ID NO:3 is a human antibody sequence from which the single domain antibody was engineered:

SASQVQLVKSEAEVKKPGSSVRVSCKASGGTISSCAISWVRQAPGQGLEW
MGGIIPMFDTTNYAQNFQGRVTITADESTSTAYMDLSSLRSEDTAVYYCA
RTYYHDTSDNDGTYGMDVWGQGTTVTVSSASTKGPSGILGSGGGGSGGGG
SGGGGSQPVLTQSPSASGTPGQRVTISCSGSTSNIGNNAVNWFQQFPGKA
PKLLVYYDDLLPSGVSDRFSGSKSGTSASLAISGLQSEDEADYYCATWDD
SLNGWVFGGGTKVTVLS.

SEQ ID NO:4: is a human antibody sequence from which the single domain antibody was engineered:

SASQVQLVESEAEVKKPGSSVRVSCKASGGTISSCAISWVRQAPGQGLEW
MGGIIPMFDTTNYAQNFQGRVTITADESTSTAYMDLSSLRSEDTAVYYCA
RTYYHDTRDNDGTYGMDVWGQGTTVTVSSASTKGPSGILGSGGGGSGGGG
SGGGGSQPVLTQSPSASGTPGQRVTISCSGSSSNIGSNTVNWFQQLPGTA
PELLMYYDDLLASGVSDRFSGSKSGTSASLAISGLQSEDEGDYYCASWDD
NLNGWVFGGGTKLTVLS.
SEQ ID NO: 5 is forward primer:
cgacgattgaaggtagatacccatacgacgttccagactacgc
tctgcag.
SEQ ID NO:6 is reverse primer:
cagatctcgagctattacaagtcttcttcagaaataagcttttgttc.
SEQ ID NO:7 is forward sequencing primer:
gttccagactacgctctgcagg.
SEQ ID NO:8 is reverse sequencing primer:
gattttgttacatctacactgttg.

SEQ ID NO:9 is a peptide consisting of the first 20 amino acids of htt MATLEKLMKAFESLKSFQQQ-biotin.

SEQ ID NO:10 is the amino acid sequence of V_{L}12.3

MGSQPVLTQSPSVSAAPRQRVTISVSGSNSNIGSNTVNWIQQLPGRAPEL
LMYDDDLLAPGVSDRFSGSRSGTSASLTISGLQSEDEADYYAATWDDSLN
GWVFGGGTKVTVLSGHHHHHH.

### DETAILED DESCRIPTION OF THE INVENTION

The methods and compositions of the invention in some aspects involve antibodies and antigen-binding fragments thereof that bind to a target protein, e.g., Htt protein. These antibodies or antigen-binding fragments thereof are useful as markers for proteins, for example Htt protein. The antibodies and antigen-binding fragments thereof of the invention are also useful as modulators of protein activity, for example, as inhibitors of Htt aggregation. Some antibodies of the invention will specifically bind to their target protein and interfere with the activity of the protein, and other antibodies of the invention may specifically bind to their target protein but not interfere with the protein's activity. The latter antibodies are useful in that they may be used in methods to monitor the location and form of their target proteins. As used herein, the term “target protein” means the protein to which an antibody or antigen binding fragment thereof of the invention specifically binds. As used herein, the terms “protein”, “polypeptide”, and “peptide” are used interchangeably. Examples of target proteins to which some of the single-domain antibodies of the invention specifically bind include, but are not limited to, htt, tau, β amyloid (Aβ), and alpha-synuclein.

In some embodiments of the invention, an antibody or antigen-binding fragment thereof of the invention may modulate its target protein. In some embodiments, modulating the activity of a protein may be modulating the level, stability, and/or activity of a protein associated with a disease or disorder. In these embodiments, the level of expression, functional activity, and/or stability of one or more proteins that are associated with the disease or disorder may be modulated using methods such as administration of antibodies, or nucleic acids that encode the antibodies, to inhibit activity of, or to stabilize the proteins or complexes of one or more of the proteins.

Modulating activity of a target protein can be increasing or decreasing the activity of the target protein versus a control level of activity of that protein in a reaction mixture, cell, tissue, or subject. The inhibition of activity results in a decrease in the level of activity versus a control level of activity of the protein in a reaction mixture, cell, tissue, or subject. The enhancement of activity results in an increase in the level of activity versus a control level of activity of the protein in a reaction mixture, cell, tissue, or subject.

An example, although not intended to be limiting, of a protein to which an antibody or antigen-binding fragment thereof of the invention may specifically bind is Htt protein. Some antibodies of the invention may specifically bind Htt and may be used to determine the presence of Htt aggregates and/or the intracellular location of an Htt protein. The presence of Htt may be determined using antibodies of the invention that specifically bind Htt. The antibodies may or may not modulate the activity of Htt protein. In addition, antibodies of the invention that modulate the activity of the Htt protein can be administered to a cell or subject to inhibit the activity of Htt protein. In some embodiments, the activity of the Htt is the formation of Htt aggregates.

The physiological processes associated with Huntington's disease include the formation of insoluble aggregates that include Htt protein fragments. Antibodies of the invention have been found to reduce the aggregation of Htt protein and to reduce the cellular burden of the Htt aggregations, thereby protecting cells (e.g. neurons) from Htt-induced cellular toxicity.

As used herein, the terms “huntingtin protein” and “Htt protein” mean a mutant huntingtin protein that contains one or more expanded polyglutamine regions. As used herein, the term “non-mutant huntingtin protein” means the normal, control, wild-type form of a huntingtin protein, i.e., one that does not contain an expanded polyglutamine region that contributes to Huntington's disease. It will be understood that the number of glutamine repeats present in a huntingtin protein can vary from subject to subject but the huntingtin protein will still be considered to be mutant huntingtin protein because it has an expanded polyglutamine region as compared to a normal, non-mutant polyglutamine protein. For example, non-mutant huntingtin protein encoded by DNA with from about 10 to about 35 copies of CAG will have a polyglutamine stretch, but a Htt protein encoded by DNA with more than about 35 copies of CAG will have an expanded polyglutamine stretch and is a mutant Htt protein. One of ordinary skill will be able to determine whether the number of polyglutamines in a protein is a number that indicates the protein is a mutant or non-mutant Htt protein. A mutant polyglutamine protein has abnormal function and/or activity or an additional activity or function as compared to the non-mutant polyglutamine protein, (e.g., aggregation, aggregation with transcription factors, etc.).

The methods described herein may be carried out on a subject or a sample obtained from a subject. As used herein, the term “subject” means any mammal that may or may not be in need of treatment with an antibody of the invention. For example, a control subject may be individual that is free of the disease associated with the target protein. Subjects include but are not limited to: humans, non-human primates, cats, dogs, sheep, pigs, horses, cows, rodents such as mice, hamsters, and rats. The samples used herein are any cell, body tissue, or body fluid sample obtained from a subject or from culture. In some embodiments, the cell or tissue sample includes neuronal cells and/or is a neuronal cell or tissue sample.

The biological sample can be located in vivo or in vitro. For example, the biological sample can be a tissue in vivo and an antibody of the invention that specifically binds to a protein associated with a disease, such as Huntington's disease, Parkinson's disease, or Alzheimer's disease, can be used to detect the presence of such molecules in the tissue (e.g., for imaging portions of the tissue that include the target protein). An example of such a use, though not intended to be limiting, is the use of an antibody that specifically binds Htt to detect the presence or location of Htt in a subject. Alternatively, the biological sample can be located in vitro (e.g., a biopsy such as a tissue biopsy or tissue extract or a reaction mixture, e.g. containing recombinant proteins). In a particularly preferred embodiment, the biological sample can be a cell-containing sample. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, a cerebrospinal fluid sample, a tissue biopsy, including punch biopsy, a tumor biopsy, a bodily fluid, a tissue, or a tissue extract or other methods. Samples also can be cultured cells, tissues, or organs.

Particular subjects to which the present invention can be applied are subjects at risk for or known to have a disease that is associated with the protein that is a target protein for the antibody of the invention. Examples of diseases, though not intended to be limiting include neurological diseases. Neurological diseases, to which the methods and compositions of the invention can be applied include, but are not limited to, Huntington's disease, Parkinson's disease, and Alzheimer's disease. One of ordinary skill will recognize that the antibodies of the invention can be applied for the treatment and/or diagnosis of many additional diseases, including, but not limited to HIV and cancer. The high affinity, high level expression, and ability to express functional antibody or antigen-binding fragments thereof in cells, tissues, and/or subjects permits their use in numerous conditions that involve a target protein to which the antibody or antigen-binding fragment thereof specifically binds. The antibodies and methods of the invention are useful diagnostically and/or therapeutically in advance of as well as after the onset of any clinical and/or physiological manifestation of a disease, e.g. symptoms of a disease. Antibodies and/or antigen-binding fragments thereof, that specifically bind to a target protein (e.g. Htt protein), are useful in therapeutic, diagnostic, pharmaceutical development, and screening methods of the invention.

As used herein, antibodies of the invention include single domain antibodies (e.g. V_{L}S), and antibodies that are disulfide-independent antibodies. As used herein, the term “disulfide-independent” means antibodies without disulfide bonds, antibodies engineered with disulfide bonds removed (whether or not the disulfide bonds are reintroduced), and/or antibodies that maintain engineered affinity level in the presence or absence of cysteine amino acids under reducing conditions. In some embodiments, a disulfide-independent antibody or antigen-binding fragment thereof of the invention is a disulfide-free antibody or antigen-binding fragment thereof. In some embodiments, a disulfide-independent antibody is an antibody that has been engineered such that its affinity does not decrease by more than 10-fold when either of the cysteines are mutated to a different residue or when both cysteines are present but under reducing conditions. A disulfide-independent antibody of the invention is an antibody that has no significant loss of affinity in the absence of a disulfide bond, where loss of the disulfide bond is either due to synthesis under reducing conditions or due to genetic substitution of one or both cysteine residues.

The single-domain, disulfide-independent antibodies of the invention may also be referred to herein as “intrabodies”. The term “intrabody” is an art-recognized term that includes intracellularly expressed antibodies. In some embodiments, the single domain antibodies are disulfide-free antibodies. As described herein, the antibodies of the present invention may be prepared by starting with any of a variety of methods, including administering protein, fragments of protein, cells expressing the protein or fragments thereof and the like to an animal to induce polyclonal antibodies. The production of monoclonal antibodies is well known in the art. As detailed herein, such antibodies or antigen-binding fragments thereof may be used in the preparation of scFvs, V_{L}S and disulfide-free variants thereof. Additional steps in the production of antibodies of the invention include directed antibody evolution and affinity engineering, as described in the Examples section.

The disulfide-independent, single-domain antibodies, and antigen-binding fragments thereof of the invention have an binding affinity (Kd) that in some embodiments, is between about 50 nM and about 5 nM. In some embodiments, the affinity of a disulfide-independent, single-domain antibody or antigen-binding fragment thereof of the invention is about 10 nM. In some embodiments, the affinity of a disulfide-independent, single-domain antibody or antigen-binding fragment thereof of the invention is between about 5 nM and 3 nM. In some embodiments, the affinity of a disulfide-independent, single-domain antibody or antigen-binding fragment thereof of the invention is less than about 3 nM. In certain embodiments, a disulfide-independent, single-domain antibody or antigen-binding fragment thereof of the invention may have a Kd value greater than about 50 nM. The use of an antibody or antigen-binding fragment thereof of the invention that has a Kd value above about 50 nM, between about 50 nM and 5 nM, between about 5 nM and 3 nM, or below about 3 nM can be determined by one of ordinary skill in the art using methods provided herein and/or art-known antibody activity assay methods.

The term “directed evolution” is an art-recognized term that describes a set of techniques for the iterative production, evaluation, and selection of variants of a biological sequence, usually a protein or nucleic acid. Directed evolution methods include, but are not limited to, the use of display methods. Display techniques that can be used in the directed evolution methods of the invention include, but are not limited to, phage display (see Hoogenboom et al., Immunol Today Aug. 21, 2000(8):371-8), single chain antibody display (see Daugherty et al., Protein Eng Jul. 12, 1999 (7):613-21; Makeyev et al., FEBS Lett Feb. 12, 1999 ;444(2-3):177-80), retroviral display (see Kayman et al., J Virol March 1999;73(3):1802-8), bacterial surface display (see Earhart, Methods Enzymol 2000;326:506-16), yeast surface display (see Shusta et al., Curr Opin Biotechnol April 1999;10(2):117-22 and U.S. patent application No. 20040146976), and ribosome display (see Schaffitzel et al., J Immunol Methods Dec. 10,1999 ;231(1-2):119-35).

Additional directed evolution methods that are useful in the methods of the invention include various mutagenesis methods such as DNA shuffling and error-prone PCR to generate mutations in antibody sequences. Directed evolution methods that are useful in the methods of the invention also include affinity maturation methods, which may be followed by the testing for affinity the antibody for the target protein. Examples of directed evolution methods such as display methods, DNA shuffling, error-prone PCR, and affinity maturation methods are provided in the Examples section. Examples of directed evolution methods are also provided in U.S. Pat. Nos. 6,489,145, 6,713,279, 6,479,258, and 6,174,673.

As detailed herein, the antibodies or antigen-binding fragments thereof may be used for example to identify a target protein and/or to modulate the activity of a target protein (e.g. as described for Htt). Using methods described herein, antibodies or antigen-binding fragments thereof can be identified and utilized that bind specifically to target proteins such as Htt protein. As used herein, “binding specifically to” means capable of distinguishing the identified material from other materials sufficient for the purpose to which the invention relates. Thus, “binding specifically to” a target protein means the ability of the antibody or antigen-binding fragment thereof to bind to and distinguish the target protein from other proteins. In some embodiments, an antibody or antigen-binding fragment thereof may bind specifically to a complex that includes one or more polypeptides that are associated with the target protein, for example a complex that includes Htt protein and/or transcription factors etc.

Antibodies of the invention may be coupled to specific diagnostic labeling agents, for imaging of cells and tissues, or to therapeutically useful agents according to standard coupling procedures. A wide variety of detectable labels can be used, such as those that provide direct detection (e.g., radioactivity, luminescence, fluorescence, optical or electron density, etc.) or indirect detection (e.g., epitope tag such as the FLAG epitope, enzyme tag such as horse-radish peroxidase, etc.). A variety of methods may be used to detect the label, depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-1 8 and carbon-I1, gamma emitters such as iodine-1 23, technitium-99m, iodine-131 and indium-111, nuclides for nuclear magnetic resonance such as fluorine and gadolinium. Other diagnostic agents useful in the invention will be apparent to one of ordinary skill in the art.

In some embodiments, the antibodies or antigen-binding fragments may be coupled to cytotoxic agents, including, but not limited to, methotrexate, radioiodinated compounds, toxins such as ricin, other cytostatic or cytolytic drugs, and so forth. Additional suitable chemical toxins or chemotherapeutic agents that may be coupled to antibodies include members of the enediyne family of molecules, such as chalicheamicin and esperamicin. Cytotoxic radionuclides or radiotherapeutic isotopes may be alpha-emitting isotopes such as ²²⁵Ac, ²¹¹At, ²¹²Bi, or ²¹³Bi. Alternatively, the cytotoxic radionuclides may be beta-emitting isotopes such as ¹⁸⁶Rh, ¹⁸⁸Rh, ⁹⁰Y, ¹³¹I or ⁶⁷Cu. Further, the cytotoxic radionuclide may emit Auger and low energy electrons such as the isotopes ¹²⁵I, ¹²³I or ⁷⁷Br. Other chemotherapeutic and radiotherapeutic agents are known to those skilled in the art.

Antibodies or antigen-binding fragments thereof of the invention that bind to a target protein or fragment thereof include antibodies prepared according to the methods provided in the Examples section herein or prepared as described elsewhere herein. Such antibodies include, but are not limited to: antibodies that bind specifically to a target protein that is associated with a disorder, antibodies that bind specifically to fragments of a target protein that is associated with a disorder, and antibodies that bind to complexes of target proteins or fragments thereof that are associated with a disorder. An example, although not intended to be limiting, of such a target protein and target protein-associated disorder is Htt protein and HD, respectively. Antibodies of the invention may be single domain antibodies. In preferred embodiments, of the invention, the antibodies are disulfide-free antibodies.

The antibodies and antigen-binding fragments thereof of the invention may be developed from antibodies identified that specifically bind to an epitope on a target protein. Significantly, as is well known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F9(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (Frs), which maintain the tertiary structure of the paratope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

As in known in the art, antibody fragments also include F(ab′)₂, Fab, Fv and Fd fragments; chimeric antibodies in which the Fc and/or Fr and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences.

The invention involves, in part, single domain antibodies or antigen-binding fragments thereof of numerous sizes and types that bind specifically to a target protein or fragment thereof or a complex of target proteins or fragments thereof, which are associated with a disorder. These polypeptides may be derived using methods set forth in the Examples section and may also be derived using other methods of antibody technology known to those of skill in the art.

The antibodies useful for practicing the invention can be initially be isolated and/or developed from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, also can be synthesized chemically using well-established methods of peptide synthesis.

Thus, as used herein with respect to antibodies, “isolated” means separated from its native environment and present in sufficient quantity to permit its identification or use. Isolated, when referring to an antibody sequence, means, for example: (i) selectively produced by expression of a recombinant nucleic acid or (ii) purified as by chromatography or electrophoresis.

Isolated antibodies may, but need not be, substantially pure. The term “substantially pure” means that the antibodies are essentially free of other substances with which they may be found in nature or in in vivo systems to an extent practical and appropriate for their intended use. Substantially pure antibodies may be produced by techniques well known in the art. Because an isolated antibody may be admixed with a pharmaceutically acceptable carrier in a pharmaceutical preparation, the antibody may comprise only a small percentage by weight of the preparation. The antibody is nonetheless isolated in that it has been separated from the substances with which it may be associated in living systems.

The invention also includes in some aspects the use of sequence related to the sequences that encode the antibodies of the invention (e.g. SEQ ID NO:1, 2, 3, 4, and 10). In addition, the invention also includes recombinant antibodies that include an amino acid sequence from the sequences set forth as SEQ ID NO:1, 2, 3, 4, and 10. These sequences can be cloned into additional antibody backgrounds to make other types of antibodies [e.g. Fab, f(ab′)₂, etc.] as described above herein. The invention also includes the nucleic acid sequences that encode the polypeptide sequences of the invention. Thus, the invention includes the nucleic acids encoding the sequences set forth as SEQ ID NOs:1-4, and 10).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating antibody polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides that are useful for practicing the invention). In preferred embodiments, these modified nucleic acid molecules and/or the polypeptides they encode retain at least one activity or function of the unmodified nucleic acid molecule and/or the polypeptides, such as binding to the target protein, inhibition of Htt activity, etc.

In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under high stringency conditions known to one of skill in the art.

For example, modified nucleic acid molecules that encode polypeptides having single amino acid changes can be prepared. Each of these nucleic acid molecules can have 1, 2, 3, 4, 5, 6, 7, 8, 9, 10, 11, 12, 13, 14, 15 or more nucleotide substitutions exclusive of nucleotide changes corresponding to the degeneracy of the genetic code as described herein. Preparation of modified nucleic acids molecules that have amino acid changes are demonstrated in the Examples section herein, which includes examples showing the use of substitutions in the preparation of antibodies of the invention. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The skilled artisan will also realize that conservative amino acid substitutions may be made in the antibodies of the invention to provide functionally equivalent variants, or homologs of the foregoing antibodies, i.e, the variants retain the functional capabilities of the antibodies. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the antibody sequences include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. For example, upon determining that a antibody binds a specific target protein, one can make conservative amino acid substitutions to the amino acid sequence of the antibody, and still have the antibody retain its specific binding characteristics.

Conservative amino-acid substitutions in the amino acid sequence of antibodies of the invention to produce functionally equivalent variants of the antibodies typically are made by alteration of a nucleic acid the antibody amino acid sequences. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a target binding antibody of the invention. Where amino acid substitutions are made to a small unique fragment of an antibody, the substitutions can be made by directly synthesizing the peptide. The activity of functionally equivalent fragments of antibodies of the invention can be tested by cloning the gene encoding the altered antibody into a bacterial or mammalian expression vector, introducing the vector into an appropriate host cell, expressing the altered antibody, and testing for a functional capability of the antibody as disclosed herein. Peptides that are chemically synthesized can be tested directly for function, e.g., for inhibiting activity of a target protein.

The invention also relates in part to the use of methods and/or compounds to prevent and/or treat diseases associated with a target protein, (e.g. HD, which is associated with the target protein, Htt), and/or manifestations of such diseases. Antibodies of the invention that are useful for the prevention and/or treatment of a disease associated with a target protein (e.g. HD) include compounds that modulate the activity of the target protein. For example, an antibody of the invention that is useful for the treatment or prevention of HD is an antibody that specifically binds to the target protein Htt, and inhibits its aggregation activity. The inhibition or enhancement of the activity of a target protein may be modulated using an antibody of the invention. Another example of an antibody that is useful to modulate activity of its target protein is an antibody of the invention that binds to β amyloid (Aβ) and modulates its aggregation. In some embodiments, an antibody of the invention may modulate the stability and/or activity of the target protein.

The invention also provides antibodies for use in methods to modulate the activity of target proteins. In such methods, the antibodies recognize and bind specifically to a protein, a fragment thereof, and/or a complex of proteins that is associated with the target protein. The binding of the antibody enhances or inhibits activity of the target protein. For example, methods to modulate (increase or decrease) the level of activity of the Htt protein may be used to prevent or treat a polyglutamine expansion-associated disease such as Huntington's disease.

As used herein, the term “modulate” means to change, which in some embodiments means to enhance and in other embodiments, means to inhibit. In some embodiments, the activity of a target protein is enhanced. In some embodiments, stabilization or activity of a target protein is increased. It will be understood that increase may mean an increase to any level that is significantly greater than the original level or a control level. In certain embodiments, that activity of a target protein is inhibited. In some embodiments, stabilization or activity of a target protein decreased. It will be understood that decrease may mean a decrease to any level that is significantly less than the original level or a control level.

In some embodiments, a single-domain antibody of the invention can modulate the level of activity of the target protein by decreasing the target protein activity by greater than 0.1%, greater than 0.2%, greater than 0.5%, greater than 1.0%, 2.0%, 3.0%, 4.0%, 5.0%, 7.0%, 10%, 15%, 20%, 25%, 30%, 40%, 50%, or more compared to the starting level. It will be understood that in other embodiments, where a single domain antibody of the invention may act to increase its target protein's activity by greater than 0.1%, greater than 0.2%, greater than 0.5%, greater than 1.0%, 2.0%, 3.0%, 4.0%, 5.0%, 7.0%, 10%, 15%, 20%, 25%, 30%, 40%, 50%, or more compared to the starting level.

The methods of the invention include the contacting intracellularly a cell in a sample or subject with an antibody or antigen-binding fragment thereof to detect the target protein and/or to modulate the activity of the target protein. Thus, the invention includes in some embodiments, methods for intracellular delivery of the antibodies.

Various forms of the antibody polypeptide sequence or encoding nucleic acid, as described herein, can be administered and delivered to a mammalian cell (e.g., by virus or liposomes, as naked DNA, or by any other suitable methods known in the art or later developed). The small size of each transcriptional unit of a single domain antibody of the invention may allow for the concatenation of multiple intrabody specificities within a single plasmid or virus. The method of delivery can be modified to target certain cells, and in particular, cell surface receptor molecules or antigens present on neuronal cells and/or other specific cell types. Methods of targeting cells to deliver nucleic acid constructs are known in the art. The antibody polypeptide sequence can also be delivered into cells by expressing a recombinant protein fused with peptide carrier molecules. These carrier molecules, which are also referred to herein as protein transduction domains (PTDs), and methods for their use, are known in the art. Examples of PTDs, though not intended to be limiting, are tat, antennapedia, and synthetic poly-arginine. These delivery methods are known to those of skill in the art and are described in U.S. Pat. No. 6,080,724, and U.S. Pat. No. 5,783,662, the entire contents of which are hereby incorporated by reference.

Methods for delivery may also include the use of expression vectors that can be delivered into cells. In some embodiments, the expression vectors include sequences that encode an antibody or antigen-binding fragment thereof of the invention. In some embodiments, sequences that encode more than one antibody or antigen-binding fragment thereof may be include in an expression vector. In some embodiments, the expression vectors may be used to transfect host cells and cell lines, be these prokaryotic (e.g., E. coli), or eukaryotic (e.g., CHO cells, COS cells, yeast expression systems and recombinant baculovirus expression in insect cells). Especially useful are mammalian cells such as human, mouse, hamster, pig, goat, primate, etc. They may be of a wide variety of tissue types, and they may be primary cells or cell lines. The expression vectors require that the pertinent sequence, i.e., those nucleic acids described supra, be operably linked to a promoter.

According to yet another aspect of the invention, an antibody or antigen-binding fragment thereof may be delivered to a cell using an expression vector. In some embodiments of the invention, expression vectors comprising any of the isolated nucleic acid molecules that encode any of the polypeptides of the invention, preferably operably linked to a promoter are provided. In a related aspect, host cells transformed or transfected with such expression vectors also are provided. Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a protein of the invention, fragment, or variant thereof. The heterologous DNA (RNA) is placed under operable control of transcriptional elements to permit the expression of the heterologous DNA in the host cell.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., β-galactosidase or alkaline phosphatase), and genes that visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

As used herein, the term “expression vectors” also includes transfer and delivery vectors. Thus, viral vectors that can be used in the methods of the invention to transfer (deliver) nucleic acid molecules are referred to herein as expression vectors.

In some embodiments, a virus vector for delivering a nucleic acid molecule encoding an antibody or antigen-binding fragment thereof of the invention is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

Preferably the foregoing nucleic acid delivery vectors: (1) contain exogenous genetic material that can be transcribed and translated in a mammalian cell and that can suppress target protein-associated disorders, and preferably (2) contain on a surface a ligand that selectively binds to a receptor on the surface of a target cell, such as a mammalian cell, and thereby gains entry to the target cell.

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like.

In addition to delivery through the use of vectors, nucleic acids of the invention (e.g. nucleic acids that encode a polypeptide of the invention) may be delivered to cells without vectors, e.g. as “naked” nucleic acid delivery using methods known to those of skill in the art.

The prevention and treatment methods of the invention include administration of the antibodies or antigen-binding fragments thereof of the invention that modulate the activity of the target protein. Various techniques may be employed for introducing an antibody or antigen-binding fragment thereof of the invention to cells, depending on whether the compounds are introduced in vitro or in vivo in a host. In some embodiments, the target protein of an antibody or antigen-binding fragment thereof is in a specific cell or tissue type, e.g. neuronal cells and/or tissues. Thus, the antibody or antigen-binding fragment thereof can be specifically targeted to neuronal tissue (e.g., neuronal cells) using various delivery methods, including, but not limited to: administration to neuronal tissue, the addition of targeting molecules to direct the compounds of the invention to neuronal cells and/or tissues. Additional methods to specifically target molecules and compositions of the invention to brain tissue and/or neuronal tissues are known to those of ordinary skill in the art.

In some embodiments of the invention, an antibody or antigen-binding fragment thereof of the invention may be delivered in the form of a delivery complex. The delivery complex may deliver the antibody or antigen-binding fragment thereof into any cell type, or may be associated with a molecule for targeting a specific cell type. Examples of delivery complexes include a antibody or antigen-binding fragment thereof of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell specific receptor). Some delivery complexes may be sufficiently stable in vivo to prevent significant uncoupling prior to internalization by the target cell. However, the delivery complex can be cleavable under appropriate conditions within the cell so that the antibody or antigen-binding fragment thereof is released in a functional form.

An example of a targeting method is the use of liposomes to deliver an antibody or antigen-binding fragment thereof of the invention into a cell. Liposomes may be targeted to a particular tissue, such as neuronal cells, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

Liposomes are commercially available from Invitrogen, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic compound (agent) of the invention, increasing convenience to the subject and the physician. Many types of release delivery systems are available and known to those of ordinary skill in the art. They include polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”. PCT/US/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in PCT/US/03307. The polymeric matrix preferably is in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers that can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of established neurological disorder conditions as well as subjects at risk of developing a neurological disorder. “Long-term” release, as used herein, means that the implant is constructed and arranged to deliver therapeutic levels of the active ingredient for at least 7 days, preferably 30-60 days, and more preferably several months or years. The implant may be positioned at or near the site of the tissue that contains the target protein. In neurological diseases, the region for the implant may be the area of neurological damage or the area of the brain or nervous system affected by or involved in the neurological disorder. Long-term sustained release implants are well known to those of ordinary skill in the art and include some of the release systems described above.

Some embodiments of the invention include methods for treating a subject to reduce the risk of manifesting a disorder associated with activity of a target protein. The methods involve selecting and administering to a subject who is known to have, is suspected of having, or is at risk of having disorder associated with abnormal activity of a target protein, an antibody or antigen-binding fragment thereof of the invention for treating the disorder. Preferably, the antibody or antigen-binding fragment thereof for modulating activity of a target protein associated with a disease is administered in an amount effective to modulate (increase or decrease) levels of the target protein activity. For example, preferably the antibody or antigen-binding fragment thereof for modulating Htt activity is administered in an amount effective to modulate (decrease) the aggregation activity of the Htt protein.

Another aspect of the invention involves reducing the risk of manifesting a disorder associated with abnormal activity of a target protein using treatments and/or medications to modulate levels of activity of the target protein, therein reducing, for example, the subject's risk of a the target-protein associated disease or disorder.

In a subject determined to have a target protein-associated disease, an effective amount of an antibody or antigen-binding fragment thereof is that amount effective to modulate (e.g. increase of decrease) the activity of the target protein associated with the disease. For example, in the case of Huntington's disease an effective amount of an antibody or antigen-binding fragment thereof of the invention may be an amount that decreases the aggregation of Htt in the subject. In disease instances in which the abnormal activity of the target protein is a level lower than that in a disease-free sample, an effective amount may be an amount that increases (enhances) the activity of the target protein.

A response to a prophylatic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has a target protein-associated disease, e.g. Huntington's disease, Alzheimer's disease, Parkinson's disease, etc., and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has modulated the activity of the target protein.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to modulate the activity of the target protein and reduce, prevent, or eliminate the target protein-associated disorder and/or its symptoms. Such determinations are considered routine for those of skill in the medical arts. For example, testing can be performed to determine the level of Htt aggregation in a subject's tissue and/or cells. Additional tests useful for monitoring the onset, progression, and/or remission (regression) of a target protein-associated disease such as those described above herein, are well known to those of ordinary skill in the art. As would be understood by one of ordinary skill, for some disorders (e.g. Huntington's disease) an effective amount would be the amount of a pharmacological agent of the invention that decreases the activity of the target protein (Htt aggregation) to a level that diminishes the disease, as determined by the aforementioned tests. For other diseases, similar strategies can be used to determine an effective amount through the monitoring of the onset progression and or regression or a target-protein-associated disorder.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

The pharmaceutical compositions used in the foregoing methods preferably are sterile and contain an effective amount of a pharmacological agent for producing the desired response in a unit of weight or volume suitable for administration to a patient. The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent of the invention may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

Various modes of administration will be known to one of ordinary skill in the art which effectively deliver the pharmacological agents of the invention to a desired tissue, cell, or bodily fluid. The administration methods include: topical, intravenous, oral, inhalation, intracavity, intrathecal, intrasynovial, buccal, sublingual, intranasal, transdermal, intravitreal, subcutaneous, intramuscular and intradermal administration. The invention is not limited by the particular modes of administration disclosed herein. Standard references in the art (e.g., Remington 's Pharmaceutical Sciences, 20th Edition, Lippincott, Williams and Wilkins, Baltimore Md., 2001) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration (e.g., intra-organ) and the like vary from those presented herein.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases. Thus, this invention is intended to be used in husbandry and veterinary medicine as well as in human therapeutics.

When administered, the pharmaceutical preparations of the invention (e.g. preparations that include antibodies or antigen-binding fragments thereof of the invention) are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. Preferred components of the composition are described above in conjunction with the description of the pharmacological agents and/or compositions of the invention.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal. The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid may be used in the preparation of injectables. Carrier formulation suitable for oral, subcutaneous, intravenous, intramuscular, etc. administrations can be found in Remington 's Pharmaceutical Sciences, Mack Publishing Co., Easton, Pa.

In general, the treatment methods involve administering an antibody to modulate the level of activity of a target protein associated with a disease. Thus, in certain embodiments, the treatment methods include gene therapy applications. The procedure for performing ex vivo gene therapy is outlined in U.S. Pat. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements, which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes also is contemplated according to the invention.

In certain embodiments, the method for treating a subject with a target protein activity associated disorder involves administering to the subject an effective amount of a nucleic acid molecule to treat the disorder. In certain embodiments, the method for treatment involves administering to a subject an effective amount of a nucleic acid that encodes the sequence of the antibody or antigen-binding fragment thereof that modulates the activity of the target protein in an amount sufficient to treat the disorder. Thus, the invention relates in part to treatment methods that involve administering to the subject an effective amount of an antibody, or antigen-binding fragment thereof to modulate an activity of the target protein and, thereby treat the disorder. In some embodiments, the treatment method involves administering to the subject an effective amount of an antibody or antigen-binding fragment thereof to increase the activity of a target protein associated the disease. In other embodiments, the treatment method involved administering to the subject an effective amount of an antibody or antigen-binding fragment thereof to decrease the activity of a target protein associated with the disease.

The invention also involves a variety of assays based upon detecting the level of binding of an antibody of the invention to its target protein in a cell, tissue, or subject. The assays can include (1) characterizing the level or cellular localization of the target protein, (2) characterizing the proximity of the target proteins to each other (e.g. is there target protein aggregation); (3) evaluating a treatment for regulating levels and/or activity of the target protein in a cell and/or subject; and/or (4) selecting a treatment for regulating the level and/or activity of a target protein in a cell and/or subject. For example, an assay system that is useful in HD may include (1) characterizing the level or cellular localization of Htt; (2) evaluation the presence of aggregation of Htt; (3) evaluating a treatment for regulating (e.g. decreasing) levels and/or activity of Htt protein; and/or (4) selecting a treatment for regulating (e.g. decreasing) levels and/or activity of Htt protein.

The invention also includes methods to monitor the onset, progression, or regression of a disease or disorder in a subject by, for example, obtaining samples at sequential times from a subject and assaying such samples for the presence and/or absence of specific binding of a single-domain, disulfide-independent antibody of the invention with its target protein that is a marker of the condition. A subject may be suspected of having the disease or disorder or may be believed not to have the disease or disorder and in the latter case, the sample may serve as a normal baseline level for comparison with subsequent samples. It will be understood that in some embodiments of the invention, a single-domain, disulfide-independent antibody or antigen-binding fragment thereof can be administered to a subject and the level of specific binding of the antibody or antigen-binding fragment to its target protein in the subject can be used for diagnosis and staging of the disorder in the subject.

Onset of a condition is the initiation of the changes associated with the condition in a subject. Such changes may be evidenced by physiological symptoms, or may be clinically asymptomatic. For example, the onset of Huntington's disease may be followed by a period during which there may be Huntington's disease-associated physiological changes in the subject, even though clinical symptoms may not be evident at that time. The progression of a condition follows onset and is the advancement of the physiological elements of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

A marker for a disease or condition can be the specific binding of a disulfide-independent, single-domain antibody or fragment thereof of the invention. For example, the onset of Huntington's disease or Alzheimer's disease may be indicated by the appearance of such a marker(s) in a subject's samples where there was no such marker(s) determined previously. For example, if marker(s) for Alzheimer's disease are determined not to be present in a first sample from a subject, and Alzheimer's disease marker(s) are determined to be present in a second or subsequent sample from the subject, it may indicate the onset of Alzheimer's disease. It will be understood that different diseases will have different markers and that one of ordinary skill in the art will be able to determine suitable markers using routine methods. Single-domain, disulfide-independent antibodies of the invention that specifically bind to a marker for a disease or disorder an be used in the diagnostic methods of the invention.

Progression and regression of a disease or disorder may be generally indicated by the increase or decrease, respectively, of marker(s) in a subject's samples over time. For example, if marker(s) for a disease or disorder (e.g. Huntington's disease) are determined to be present in a first sample from a subject and additional marker(s) or more of the initial marker(s) for the disease or disorder are determined to be present in a second or subsequent sample from the subject, it may indicate the progression of the disease or disorder. Regression of a disorder or disease may be indicated by finding that marker(s) determined to be present in a sample from a subject are not determined to be found, or found at lower amounts in a second or subsequent sample from the subject.

The progression and regression of a disease or disorder may also be indicated based on characteristics of the marker as determined in an assay of the invention. For example, some disease or disorder-associated polypeptides may be abnormally expressed at specific stages of the disease or disorder (e.g. early-stage Alzheimer's disease-associated polypeptides; mid-stage Alzheimer's disease-associated polypeptides; and late-stage Alzheimer's disease-associated polypeptides).

In some embodiments of the invention, a disulfide-independent or single-domain antibody of the invention may be attached to a detectable label that can be used to determine the level of specific binding of a disulfide-independent or single-domain antibody of the invention to its target protein. In some embodiments, such determinations can be done in vivo and in other embodiments, the determination of the level of target protein can be done in vitro. Those of ordinary skill will be able to determine which detectable labels can be used in the methods of the invention. Examples of labels include, but are not limited to: those that provide direct detection (e.g., fluorescence, radioactivity, luminescence, optical or electron density, etc.) or indirect detection (e.g., enzyme tag such as horse-radish peroxidase, etc.).

The invention includes kits for assaying the presence of disease or disorder-associated proteins. Another example of a kit may include a disulfide-independent or single-domain antibody of the invention or antigen-binding fragment thereof, that binds specifically to a disease protein target. In some embodiments a disulfide-independent or single-domain antibody or antigen-binding fragment thereof may be detectably labeled. The antibody or antigen-binding fragment thereof, may be applied to a tissue sample from a patient with a disease or disorder and the sample then processed to assess whether specific binding occurs between the antibody and a protein or other component of the sample. In addition, the antibody or antigen-binding fragment thereof, may be administered to a subject for in vivo diagnostic use. As will be understood by one of skill in the art, such binding assays may also be performed with a sample or object contacted with an antibody that is in solution, for example in a 96-well plate or applied directly to an object surface.

The foregoing kits can include instructions or other printed material on how to use the various components of the kits for diagnostic purposes.

Thus, a subject's disease can be diagnosed and/or characterized, treatment regimens can be selected and monitored, and diseases can be better understood using the assays of the present invention. For example, the invention provides in one aspect a method for measuring the level of Htt protein aggregation in a cell and/or subject. For example, a level Htt aggregation that is significantly higher in a subject than a control level may indicate a subject has HD, whereas a relatively normal level of Htt may indicate that the subject does not have HD.

The assays described herein (see Examples section) may in some embodiments include measuring the ability of an antibody of the invention to modulate activity of a target protein and/or the ability of an antibody of the invention label a target protein thereby enabling use of the antibody to detect the target protein in a cell and/or subject. The examples provided herein demonstrate methods to determine the affinity, activity, and of the antibodies of the invention as well as to determine expression of the target proteins to which the antibodies of the invention specifically bind.

Importantly, the specific binding of an antibody or antigen-binding fragment thereof of the invention, and/or the modulation (e.g. inhibition) of a target protein's activity by an antibody or antigen-binding fragment thereof of the invention is advantageously compared to controls according to the invention. The control may be a predetermined value, which can take a variety of forms. It can be a single value, such as a median or mean. It can be established based upon comparative groups, such as in groups having normal amounts of activity of the target protein (e.g. aggregation of Htt protein). The control level may be the amount of target protein activity (e.g. Htt protein aggregation in a cell that is not contacted with an antibody or antigen-binding fragment of the invention. Other groups that can be used as a comparative group are groups having abnormal amounts or activity of a target protein (e.g. Htt protein). Another example of comparative groups would be groups having a particular disease (e.g., HD, Alzheimer's disease, Parkinson's disease, etc.), condition or symptoms, and groups without the disease, condition or symptoms. Another comparative group would be a group with a family history of a condition and a group without such a family history. The predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk the highest quadrant or quintile being individuals with the highest risk.

The predetermined value of course, will depend upon the particular population selected. For example, an apparently healthy population will have a different ‘normal’ range than will a population that is known to have a condition related to abnormal activity of a target protein. Accordingly, the predetermined value selected may take into account the category in which an individual falls. Appropriate ranges and categories can be selected with no more than routine experimentation by those of ordinary skill in the art. By abnormally high it is meant high relative to a selected control. Typically the control will be based on apparently healthy normal individuals in an appropriate age bracket. As used herein, the term “difference” or “differences” means statistically significant difference or differences.

It will also be understood that the controls according to the invention may be, in addition to predetermined values, samples of materials tested in parallel with the experimental materials. Examples include samples from control populations or control samples generated through manufacture to be tested in parallel with the experimental samples.

The various assays used to determine the specific binding of an antibody or antigen-binding fragment thereof to a target protein and/or the ability of the antibody or antigen-binding fragment thereof to modulate the activity of the target protein, include: assays, such as described in the Examples section herein, and assays such electrophoresis; NMR; and the like. Immunoassays may be used according to the invention including sandwich-type assays, competitive binding assays, one-step direct tests and two-step tests such as routinely practiced by those of ordinary skill in the art. Methods of using the antibodies of the invention to detect the location or activity of target proteins include fluorescence resonance energy transfer (FRET) methods. Examples of the use of FRET methods are provided in the Examples section. The use of FRET methods in the some aspects of the invention includes the use of reporter polypeptides. Examples of reporter polypeptides that can be used in the methods of the invention, although not intended to be limiting, include: yellow fluorescent protein (YFP), cyan fluorescent protein (CFP), β-galactosidase, chloramphenicol acetyl transferase (CAT), luciferase, green fluorescent protein (GFP). Additional FRET methods can be utilized using methods known to those of ordinary skill in the art.

As mentioned above, it is also possible to characterize the effect of an antibody or antigen-binding fragment thereof on the activity of a target protein by monitoring changes in the absolute or relative level or amount of activity of the target protein over time. For example, in HD, it is expected that administering an antibody or antigen-binding fragment thereof of the invention that decreases in the aggregation of Htt protein may correlate with decreasing severity of the disease. Similarly, in other diseases an antibody or antigen-binding fragment thereof of the invention that decreases the activity of a target protein, may correlate with the decreasing severity of the disease.

In addition, it will be understood that in certain diseases the administration of an antibody or antigen-binding fragment thereof of the invention that increases the activity of a target protein may correlate with decreasing severity of the associated disease. Similarly, in certain diseases the administration of an antibody or antigen-binding fragment thereof of the invention that decreases the activity of a target protein may correlate with increasing severity of the associated disease in the cell, tissue, or subject.

Accordingly, one can monitor any change in the activity of a target protein and its effect on the status (e.g. stage, severity, etc.) of a target protein-associated disease. Changes in relative or absolute activity of the target protein of greater than 0. 1% relative to a normal control level may indicate an abnormality. Preferably, the change in activity of the target protein that indicates an abnormality, is greater than 0.2%, greater than 0.5%, greater than 1.0%, 2.0%, 3.0%, 4.0%, 5.0%, 7.0%, 10%, 15%, 20%, 25%, 30%, 40%, 50%, or more. Other changes, (e.g. increases or reductions) in levels of activity of a target protein contacted with an antibody or antigen-binding fragment thereof of the invention, over time may indicate an onset, progression, regression, or remission of the target protein-associated disease in the cell, tissue, and/or subject. As described above, in some disorders such as HD, a decrease in the activity (e.g. aggregation) of the target protein, Htt, may mean regression of the disorder. Such a regression may be associated with a clinical treatment of the disorder. Thus, the methods of the invention can be used to determine the efficacy of a therapy for a target protein associated disorder, (e.g. HD). In some disorders an increase in the activity of a target protein by an antibody or antigen-binding fragment thereof may mean regression of the disorder.

The invention in another aspect provides a diagnostic method to determine the effectiveness of treatments for abnormal levels of target protein and/or target protein activity, e.g. abnormal levels of Htt aggregation. The “evaluation of treatment” as used herein, means the comparison of a subject's levels of target protein and/or levels of target protein activity measured in samples collected from the subject at different sample times, preferably at least one day apart. In some embodiments, the time to obtain the second sample from the subject is at least one day after obtaining the first sample, which means the second sample is obtained at any time following the day of the first sample collection, preferably at least 12, 18, 24, 36, 48, 96 or more hours after the time of first sample collection. In some embodiments, the second sample is obtained from the subject 1, 2, 3, 4, 5, 6, 7, 8, 9, 10, 11, or more hours after the first sample is obtained. In some embodiments, days, weeks, or months may pass between the time of a first sample collection or administration of an antibody of the invention and the time of a second or subsequent collection or administration. It will be understood that the multiple samples may also be obtained from cells and/or tissues in culture, thus the invention includes methods of testing treatments in vitro in addition to the methods for testing treatments and their effects in vivo.

The comparison of the level of target protein and/or target protein activity in two or more samples, taken at different times or on different days, is a measure of level of the subject's (or tissue's and/or cell's) diagnostic status for a target protein associated disorder and allows evaluation of a treatment to regulate the activity of the target protein and or the efficacy of an antibody or antigen-binding fragment thereof of the invention to modulate activity of the target protein (e.g. aggregation of Htt protein; enzyme activity; protein-protein binding). The comparison of a subject's, tissue's, and/or cell's target protein and/or target protein activity measured in samples obtained on different days provides a measure of the status of the target protein associated disorder to determine the effectiveness of any treatment to regulate the level and/or activity of the target protein in the subject, tissue, and/or cell, either by use of an antibody or antigen-binding fragment of the invention to treat the disorder or using another therapeutic method to treat the disorder.

As will be appreciated by those of ordinary skill in the art, the evaluation of a treatment also may be based upon an evaluation of the symptoms or clinical end-points of the associated disease. In some instances, the subjects to which the methods of the invention are applied are already diagnosed as having a particular condition or disease. In other instances, the measurement will represent the diagnosis of the condition or disease. In some instances, the subjects will already be undergoing drug therapy for a target protein associated disease (e.g. HD, Alzheimer's disease, Parkinson's disease, etc.), while in other instances the subjects will be without present drug therapy for a target protein associated disorder.

The invention also relates in some aspects to methods to identify pharmacological agents that modulate the activity of a target protein. Thus, an antibody of the invention that specifically binds to a target protein, but does not modulate its activity can be used to identify pharmacological agents that may modulate the target protein's activity. For example, in a control sample, cells (or an extract thereof) from a subject known to have HD, or who express mutant Htt can be contacted with an antibody of the invention to detect the presence of Htt in the cells. In a parallel test sample, cells from the subject can be contacted with a candidate pharmacological agent and the antibody to detect a change in the localization or aggregation level of Htt in the cells contacted with the candidate agent as compared to the control cells. A wide variety of assays to identify pharmacological agents that modulate target protein activity or stability can be used in accordance with the aspects of the invention, including, labeled in vitro protein-protein binding assays, electrophoretic mobility shift assays, immunoassays, cell-based assays such as two- or three-hybrid screens, transcription assays, expression assays, etc. The assay mixture comprises a candidate pharmacological agent. Typically, a plurality of assay mixtures is run in parallel with different agent concentrations to obtain a different response to the various concentrations. Typically, one of these concentrations serves as a negative control, i.e., at zero concentration of agent or at a concentration of agent below the limits of assay detection.

Candidate agents encompass numerous chemical classes, although typically they are organic compounds. In some embodiments, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

It is contemplated that cell-based assays as described herein can be performed using cell samples and/or cultured cells. Cells include cells transformed to express a target protein or polypeptide.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An assay may be used to identify candidate agents that directly or indirectly modulate the activity of a target protein. In general, the mixture of the foregoing assay materials is incubated under conditions whereby, but for the presence of the candidate pharmacological agent, modulation (e.g. enhancement or inhibition) of activity of the target protein occurs. For example, such an assay may indicate a candidate agent is useful as a therapeutic in HD if in the assay, the presence of the candidate pharmacological agent prevents aggregation of Htt protein. It will be understood that a candidate pharmacological agent that is identified as a modulating agent may be identified as reducing or eliminating the target protein activity. A reduction in activity need not be the absence of all activity, but may be a lower level of activity. Additionally, a candidate pharmacological agent that is identified as a modulating agent may be identified as increasing the target protein activity.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours. After incubation, the presence or absence and/or level of activity of a target protein is detected using an antibody of the invention utilizing any convenient method available to the user.

The invention will be more fully understood by reference to the following examples. These examples, however, are merely intended to illustrate the embodiments of the invention and are not to be construed to limit the scope of the invention.

### EXAMPLES

### Example 1

Engineered Single Domain Antibody that Inhibits Huntingtin Aggregation

Introduction

We have engineered a single-domain antibody (also referred to herein as an intrabody) for intracellular expression and binding under the reducing conditions of the cell cytoplasm. The antibody is a variable light chain of human origin, the disulfide bond has been removed and the affinity has been engineered to 10 nM. The antibody inhibits huntingtin aggregation both in a cell-free in vitro assay and in a yeast intracellular assay.

Methods

Single-Domain Antibody Inhibits Aggregation in Cell-Free Assay

To determine if a single-domain antibody (SDAb) [also referred to herein as a single-domain intrabody (SAIb)] binding the N-terminus of huntingtin could inhibit Htt aggregation, the SDAb was secreted from yeast as a His6 fusion and purified. Approximately 1 mg was obtained of the purified protein.

In this series of experiments, the effect of the engineered antibody on huntingtin aggregation was measured using light scattering. The huntingtin protein used in this experiment was GST-httex1-Q67, and its concentration was kept constant in all samples at 500 nM. The protease thrombin was used to cleave the GST and initiate aggregation. The effect of five different antibody concentrations was studied: 60 nM, 100 nM, 300 nM, 600 nM, and 1 μM. A positive control containing no antibody and a negative control for which no thrombin was added were also studied. Light scattering measurements were taken after 48 hours of incubation at 37° C.

Sample Preparation

Each sample was prepared in a total volume of 50 μl and included: 6.8 μl of GST-httex1-Q67 (such that the final concentration was 500 nM), 6.8 μl of Ab (such that the final concentrations were 60 nM, 100 nM, 300 nM, 600 nM, or 1 μM), 35.4 μl of PBS-BSA, and 1 μl of thrombin. The positive control contained 6.8 μl of GST-httex1-Q67, 42.2 μl of PBS-BSA, and 1 μl of thrombin. The negative control contained 6.8 μl of GST-httex1-Q67 and 43.2 μl of PBS-BSA. Each sample was prepared in triplicate and then placed in a well of a 96-well polypropylene PCR plate. The plate was kept in a 37° C. warm room for 48 hours of incubation.

Light Scattering Measurements

The Varian Cary Eclipse Fluorescence Spectrophotometer was used to measure the light scattering of the samples. The excitation and emission wavelength was 495 nm. 100 μl of PBS-BSA was added to each sample and mixed by pipetting a number of times. The entire sample (which was then 150 μl) was placed in the appropriate quartz cuvette to take the light scattering measurement.

Results

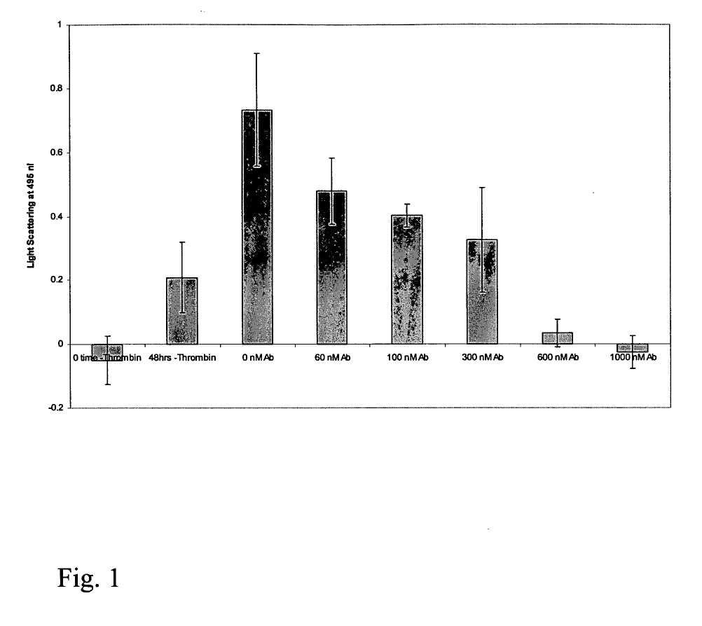

The results are shown in FIG. 1. The single domain antibody inhibited htt aggregation completely when present in stoichiometric proportion to the htt fragment (>500 nM). Since the affinity of the antibody was ˜30-50 nM, using lower concentrations of the htt fragment may result in greater aggregation inhibition at lower antibody concentrations.

Knocking out Disulfide Bond Reduces Binding Affinity

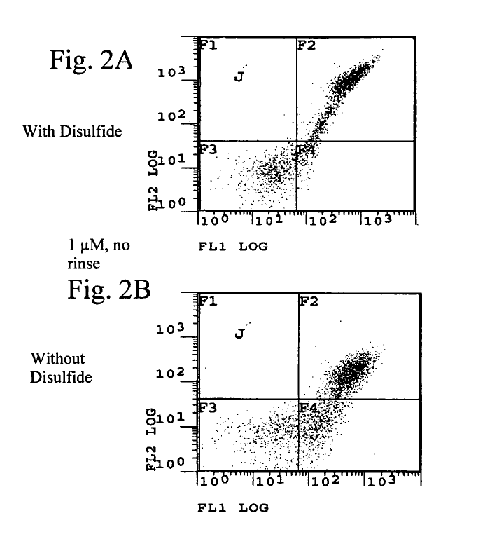

To investigate the effect of reducing conditions on the stability and binding of the SDAb, the two cysteines were mutated to valine and alanine, as described by Proba and Pluckthun (Proba K, et al., J Mol Biol. 1998 275(2):245-53.JMB, 1998). We found that the disulfide-free antibody was still well-expressed on the yeast cell surface, implying that the mutations did not severely reduce stability; however, the binding was much weaker (see FIG. 2). The affinity dropped from 30-50 nM to the micromolar range, approximately a 100-fold decrease. This implied that the antibody would not bind well under reducing conditions, and needed further affinity maturation in the absence of the disulfide bond. The results indicated that knocking out the disulfide bond reduced binding affinity, but not stability.

SDIb has Better Intracellular Expression than ScFv

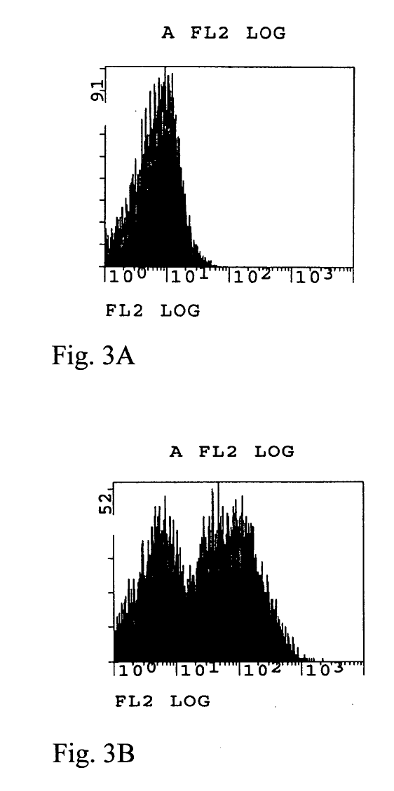

The two important parameters that determine if an antibody can bind at high levels in the cell cytoplasm are affinity and expression. To see if the antibodies we were engineering would express in the cytoplasm, we made antibody-YFP (yellow fluorescent protein) plasmid constructs for intracellular expression in yeast. The fusion protein was expressed under the control of an inducible galactose promoter. YFP fluorescence was measured 24 hours post-induction by flow cytometry (see FIG. 3). The single domain antibody was much better expressed than its scFv counterpart, possibly due to the absence of the (Gly₄Ser)₃ linker that bridges the heavy and light chains.

Disulfide-Free SDIb Affinity Matured to 10 nM

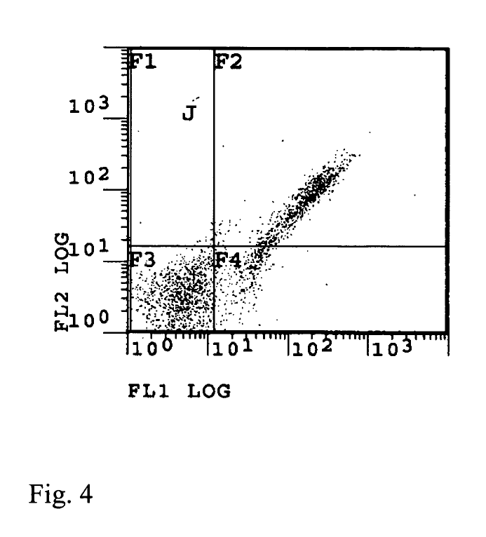

Given that the SDIb expressed well in the cytoplasm, but bound only weakly when the disulfide bond was knocked out, we then affinity matured the disulfide-free SDIb using yeast surface display, as described in Example 2. After three rounds of mutation and screening, the SDIb affinity was increased to ˜10 nM, even higher than the original scFv. FIG. 4 shows the flow cytometry data of the yeast surface displayed SDIb labeled at 1 nM huntingtin peptide. Significant fluorescence was observed even at this low peptide concentration. Seven additional amino acid mutations were acquired during the rounds of mutagenesis.

Engineered SDIb Inhibits Huntingtin Aggregation in a Yeast HD Model

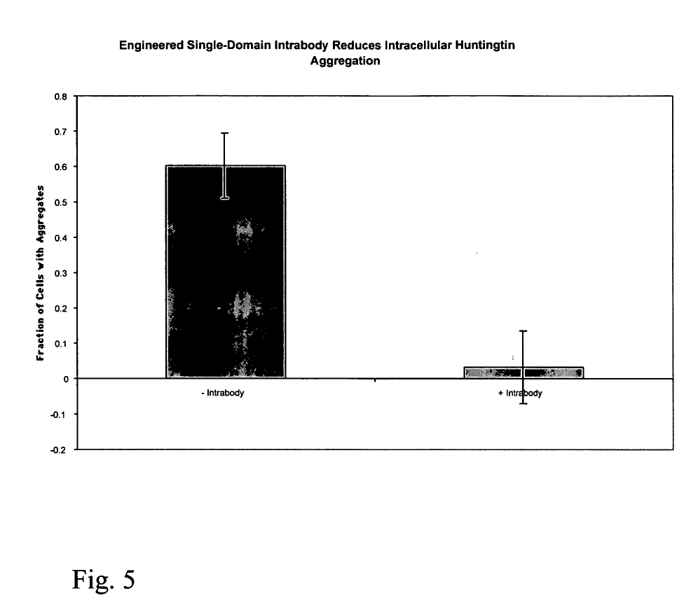

To test the engineered SDIb's ability to interfere with huntingtin aggregation, a yeast HD model was used (provided by S. Lindquist, See: Nathan, D. F., et al., Proc Natl Acad Sci USA. Feb. 16, 1999;96(4):1409-14). The SDIb was expressed cytoplasmically along with Htt-x1-Q97-YFP, which forms aggregates in the yeast cytoplasm. Images were taken on a confocal microscope, and images were analyzed quantitatively to objectively measure the fraction of cells with aggregates. Three images were taken of each sample, with approximately 50-100 cells in each image. Aggregates were scored by applying a threshold value to fluorescence intensity. Because the rate of plasmid loss can be high with non-integrated constructs, the data were corrected by assuming cells which have lost the SDIb plasmid will exhibit a level of aggregation equal to that of the negative control (no SDIb present). The result is shown in FIG. 5.

A second experiment was performed to check the reproducibility of the data, and a significant decrease in aggregation was also observed, albeit at a lower level (˜50% decrease instead of ˜90%).

Conclusions

We have developed a process of engineering a single domain antibody against the N-terminus of huntingtin. We have produced soluble SDIb and demonstrated that it was capable of inhibiting Htt aggregation in a cell-free assay. We then found that binding under reducing conditions was limited, as demonstrated by the weakened binding observed when the disulfide bond was knocked out. The SDIb was well expressed in yeast cytoplasm compared to the full scFv. The disulfide-free antibody was affinity matured to 10 nM by three additional rounds of mutation and screening. Finally, we observed significant aggregation inhibition in a yeast cell model of HD using the intrabody.

### Example 2

Yeast Surface Display (YSD)

Introduction

We provide protocols we used to engineer single chain antibodies by yeast surface display (YSD), which is a powerful tool for engineering the affinity, specificity, and stability of antibodies, as well as other proteins. Since first described six years ago by Boder and Wittrup (Boder, E. T. et al., (1997) Nat Biotechnol 15, 553-557), YSD has been employed successfully in engineering a number of antibodies (Kieke, M. C. et al., (1997) Protein Eng 10, 1303-1310; Boder, E. T. et al., (2000) Proc Natl Acad Sci USA 97, 10701-10705), as well as T-cell receptors (Holler, P. D. et al., (2000) Proc Natl Acad Sci U S A 97, 5387-5392; Kieke, M. C. et al., (1999) Proc Natl Acad Sci U S A 96, 5651-5656; Kieke, M. C. et al., (2001) J Mol Biol 307, 1305-1315). A recently reported large non-immune single chain antibody library is a good starting point for engineering high affinity antibodies (Feldhaus, M. J. et al., (2003) Nat Biotechnol 21, 163-170). Cloned variable genes from hybridomas or scFvs or Fabs from phage display libraries are also easily incorporated into a yeast display format. The original YSD protocols were described earlier (Boder, E. T. et al.,. (2000) Methods Enzymol 328, 430-444), but new and refined methods have been developed, in particular improved vectors, mutagenesis methods, and efficient ligation-free yeast transformation procedures. We provide up-to-date protocols herein, which we used to engineer single chain antibodies by YSD.

Compared to other display formats, yeast surface display offers several advantages. One chief advantage to engineering protein affinity by YSD is that yeast cells can be sorted by Fluorescence Activated Cell Sorting (FACS), allowing quantitative discrimination between mutants (VanAntwerp, J. J. et al., (2000) Biotechnol Prog 16, 31-37). Further, FACS simultaneously gives analysis data, eliminating the need for separate steps of expression and analysis after each round of sorting. Without exception to date, equilibrium binding constants and dissociation rate constants measured for yeast-displayed proteins are in quantitative agreement with those measured for the same proteins in vitro by BIAcore or ELISA. Traditional panning methods have also been employed successfully with YSD, including magnetic particle separation (Yeung, Y. A. et al., (2002) Biotechnol Prog 18, 212-220). Other advantages arising from the yeast system include ease of use and presence of the yeast endoplasmic reticulum, which acts as a quality control mechanism and ensures that only properly folded proteins reach the cell surface.

This example contains methods for displaying an antibody on yeast, creating mutant libraries, and sorting libraries for isolation of improved clones. The constructs and strains required for yeast surface display are described in the first section. The next section contains the method for creating large mutant libraries using homologous recombination, including the precise conditions used for error prone PCR using nucleotide analogues. Finally we include protocols for labeling yeast with fluorophores and sorting by FACS for improved affinity.

Methods

The Yeast Surface Display System

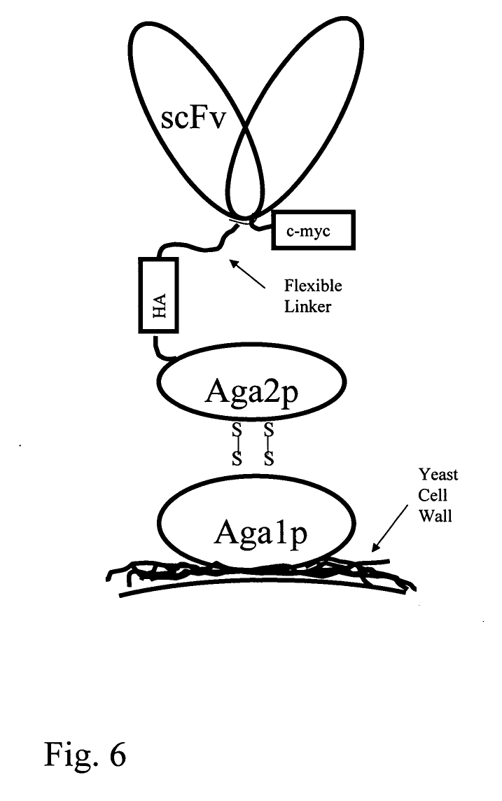

As the name implies, yeast surface display involves the expression of a protein of interest on the yeast cell wall, where it can interact with proteins and small molecules in solution. The protein is expressed as a fusion to the Aga2p mating agglutinin protein, which is in turn linked by two disulfide bonds to the Aga1p protein covalently linked to the cell wall (FIG. 6). Expression of both the Aga2p-antibody fusion and Aga1p are under the control of the galactose-inducible GAL1 promoter, which allows inducible over-expression.

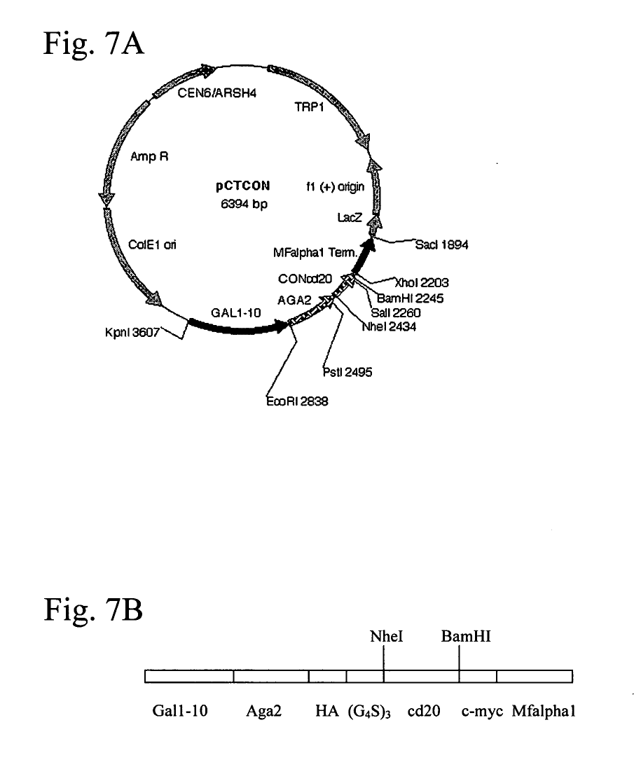

In order to use YSD, one constructs a yeast shuttle plasmid with the single-chain antibody of interest fused to Aga2p. This can be derived from the pCTCON vector (FIG. 7) by inserting the open reading frame of the scFv of interest between the NheI and BamHI sites (both of which should be in frame with the antibody). The yeast strain used must have the Aga1 gene stably integrated under the control of a galactose inducible promoter. EBY100 (Invitrogen Corp, Carlsbad, Calif.) or one of its derivatives are suggested.

Generating Large Mutant Antibody Libraries in Yeast

The most efficient way to make a mutant library in yeast is to use homologous recombination, thereby eliminating the need for ligation and E.coli transformation (Raymond, C. K. et al., (1999) Biotechniques 26, 134-8, 140-141). In brief, cut plasmid and an insert containing the mutated gene are prepared separately, with significant homology (30-50 bp or more) shared by the insert and plasmid at each end. These DNA fragments are then taken up by yeast during electroporation, and re-assembled in vivo. Libraries prepared by this method typically include at least 10⁷ transformants, and are often over 10⁸ in diversity, which approximates the amount that can be sorted by state of the art cell sorters in an hour.

In the section below herein we describe how to prepare scFv insert DNA with random point mutations by error prone PCR with nucleotide analogues. However, this may be replaced with DNA shuffling with slight modification using one of many published protocols (Stemmer, W. P. (1994) Nature 370, 389-391; Stemmer, W. P. (1994) Proc Natl Acad Sci U S A 91, 10747-10751; Volkov, A. A. et al., (2000) Methods Enzymol 328, 447-456).

Preparation of Insert: Error Prone PCR Using Nucleotide Analogues

Nucleotide analog mutagenesis allows the frequency of mutation to be tuned based on the number of PCR cycles and the relative concentration of the mutagenic analogues (Zaccolo, M. et al., (1999) E. J Mol Biol 285, 775-783; Zaccolo, M. et al., (1996) J Mol Biol 255, 589-603). The two analogues, 8-oxo-2′-deoxyguanosine-5′-triphosphate and 2′-deoxy-p-nucleoside-5′-triphosphate (8-oxo-dGTP and dPTP respectively, TriLink Biotech), create both transition and transversion mutations. In order to ensure that some fraction of the library created is sufficiently mutated to generate improvements, but not so highly mutated as to completely ablate binding, a range of several different mutagenesis levels were used in parallel. The conditions reported here are the ones we typically used to create antibody libraries; these conditions give an error rate ranging from 0.2%-5%.

If the gene to be mutated is already in pCTCON, then the following primers may be used to carry out the mutagenesis and subsequent amplification. These primers were designed to have >50 bp of homology to pCTCON for use during homologous recombination.

Forward primer: cgacgattgaaggtagatacccatacgacgttccagactacgctctgcag (SEQ ID NO:5) Reverse primer: cagatctcgagctattacaagtcttcttcagaaataagcttttgttc (SEQ ID NO: 6)

Mutagenesis and Amplification

1. Six 50 μl PCR reactions were set up as follows:

| | Final Concentration | |
|---|---|---|
| 10X PCR Buffer (without MgCl₂) | | |
| MgCl₂ | 2 | mM |
| Forward Primer | 0.5 | μM |
| Reverse Primer | 0.5 | μM |
| dNTP's | 200 | μM |
| Template | 0.1-1 | ng |
| 8-oxo-dGTP | 2-200 | μM |
| dPTP | 2-200 | μM |
| dH20 | to final volume | |
| Taq polymerase | 2.5 | units |

Of the six PCR reactions, two contained 200 μM nucleotide analogues, two contained 20 μM nucleotide analogues, and two contained 2 μM nucleotide analogues. The PCR was run for the number of cycles specified below. The cycles had the following incubation temperatures and times: denature at 94° C. for 45 sec, anneal at 55 ° C. for 30 sec, extend at 72° C. for 1 min. A 3 min denaturation step at 94 ° C. was also included before the cycles begin and a 10 min extension step was included after the cycles were completed (the 10 min extension may be done on a heating block to run all reactions simultaneously).

| Nucleotide Analogue Concentration | Number of PCR cycles |
|---|---|
| 200 μM | 5 |
| 200 μM | 10 |
| 20 μM | 10 |
| 20 μM | 20 |
| 2 μM | 10 |
| 2 μM | 20 |

The entire mutagenic PCR products were run out on a 1% low melt agarose gel. PCR products cycled 20 times were easily visible on a gel stained with SYBR Gold (Molecular Probes). Reactions cycled 10 times or less may not be visible on the gel; however, it was important to gel purify anyway to remove the non-mutated template before amplification (next step). Bands were cut out and purified using Qiagen gel purification kit (Qiagen, Valencia, Calif.) following manufacturer's protocol.

Each reaction was amplified in the absence of nucleotide analogues to generate sufficient insert DNA for the transformation. Three 100 μl reactions were set up for each mutagenic reaction, and 1 μl or more of the gel purified product was used as template in the new reaction. Nucleotide analogues were not added. The samples were cycled 25-30 times as for a normal PCR.

The following step was optionally performed. The PCR products from step 4 were gel purified. Purification eliminated many PCR artifacts from the library, but may also have resulted in significant loss of PCR product.

The PCR products were concentrated using Pellet Paint (Novagen, Inc. Madison, Wis.). After the pellet dried, the pellet was dissolved in water to a final concentration of 5 μg/μl. This protocol typically produced 40-100 μg of PCR product.

Preparation of Vector

We Prepared the Vector Using the Following Procedure:

Ten μg or more of pCTCON was minipreped. The miniprep was digested with NheI (New England Biolabs, Inc., Beverly, Mass.) for at least two hours in NEB2 buffer. The salt concentration was adjusted by adding one-tenth of the total volume of 1 M NaCl. The sample was double digested with BamHI and SalI for two additional hours, to ensure complete digestion of pCTCON and reduce reclosure of the acceptor vector. (Note that the plasmid was cut in three places to ensure that the vector will not transform yeast cells in the absence of insert.) The Qiagen nucleotide removal kit was used to purify DNA from enzymes, keeping in mind that a single column saturates with 10 μg DNA. The DNA was concentrated using Paint Pellet reagent. After drying pellet, the pellet was dissolved in water to 2 μg/μl.

Preparation of Electrocompetent Yeast Cells

This protocol was adapted from E. Meilhoc et. al. (Meilhoc, E. et al., (1990) Biotechnology (N Y) 8, 223-227), and generated enough cells for transformation of ˜60 μg of insert DNA and ˜6 μg of vector, which typically produced ˜5×10⁷ yeast transformants.

We inoculated 100 mL of YPD to OD₆₀₀ 0.1 from a fresh overnight culture of EBY100 (or appropriate yeast strain). The cells were grown with vigorous shaking at 30° C. to an OD₆₀₀ of 1.3-1.5 (about 6 hours). We added 1 mL filter sterilized 1,4-dithiothreitol (DTT, Mallinckrodt) solution (1 M tris, pH 8.0, 2.5 M DTT). DTT is unstable and the solution had to be made fresh just before use. The cells continued to grow with shaking at 30° C. for 20 min. The cells were harvested at 3500 rpm, 5 min, 4° C. and the supernatant was discarded. All centrifugation steps were carried out in autoclaved centrifuge tubes or sterile Falcon tubes. The cells were washed with 25 mL of E buffer (10 mM tris, pH 7.5, 270 mM sucrose, 1 mM MgCl₂) at room temperature, and recentrifuged to spin down cells. The cells were transferred to two 1.5 mL microcentrifuge tubes and wash a second time with 1 mL of E buffer each. The cells were recentrifuged to spin down. Both pellets were resuspended in E buffer to a final combined volume of 300 μl. Any extra cells that would not be used immediately were frozen down in 50 μl aliquots for future use. Note that using frozen cells resulted in a 3-10-fold loss in transformation efficiency.

Electroporation

Electroporation was carried out using a Biorad Gene Pulser device (BioRad Laboratories, Hercules, Calif.).

In a microcentrifuge tube, 0.5 μl vector (1 μg), 4.5 μL insert (9 μg), and 50 μL electrocompetent yeast cells were mixed. The mixture was added to a sterile 0.2 cm electroporation cuvette (Biorad). The mixture was then incubated on ice 5 min. Additional cuvettes were prepared until all of the DNA was used. The Gene pulser settings were set to 25 μF (capacitance) and 0.54 kV (voltage), which gave an electric field strength of 2.7 kV/cm with 0.2 cm cuvettes; time constant was about 18 ms with 55 μl volumes. The pulse controller accessory was not used. The pulsing was carried out at room temperature. The cuvette was inserted into the slide chamber and both red buttons were pushed simultaneously until pulsing tone was heard, then they were released. After pulsing, 1 mL of room temperature YPD media (Boder, E. T. et al.,. (2000) Methods Enzymol 328, 430-444) was immediately added to the cuvette. The mixture was incubated at 30 ° C. for 1 hour in 15 mL round bottom falcon tubes with shaking (250 rpm). The cells were spun down at 3500 rpm in a microcentrifuge. The cells were resuspended in selective media (SD+CAA, (Boder, E. T. et al., (2000) Methods Enzymol 328, 430-444) 50 mL/electroporation reaction). Serial 10-fold dilutions were plated out to determine transformation efficiency. The library could be propagated directly in liquid culture without significant bias, due to repression of scFv expression in glucose-containing medium such as SD+CAA (Feldhaus, M. J. et al., (2003) Nat Biotechnol 21, 163-170).

Transformation efficiency was at least 10⁵/μg, but was typically around 10⁶/μg. In addition to the electroporation mixture described here, we performed a control where no insert was added and determined the transformation efficiency. This was the background efficiency and was less than ˜1% of that obtained in the presence of insert DNA.

Equilibrium Labeling Protocol

Labeling yeast that were displaying an antibody or antibody library with a fluorescent or biotinylated antigen allowed quantification of binding affinity and enabled library sorting by FACS. Typically a second fluorophore conjugated to an antibody was used to detect the epitope tag C-terminal to the scFv, which allowed for normalization of expression and eliminates non-displaying yeast from quantification. The following short protocol describes labeling with a biotinylated antigen and the 9E10 monoclonal antibody against the C-terminal epitope tag c-myc. This protocol is for analytical labeling; for labeling large libraries, volumes were adjusted as describe at the end of the protocol.

Transformed yeast were grown overnight in SD+CAA. OD₆₀₀ was greater than one. As a general approximation, OD₆₀₀=1 represented 10⁷ cells/mL. A 5 mL culture of SG+CAA (Boder, E. T. et al.,. (2000) Methods Enzymol 328, 430-444) (inducing media) was inoculated with the overnight culture. The final OD₆₀₀ of the new culture was approximately 1. The culture was induced at 20° C. with shaking (250 rpm) for at least 18 hrs. Appropriate induction temperature was tested for each scFv, from 20° C., 25° C., 30° C., or 37° C. We collected 0.2 OD₆₀₀-mL of induced yeast in a 1.5 mL microcentrifuge tube. Several such aliquots were sometimes necessary to sample the full diversity of the library, since the aliquot corresponded to approximately 2×10⁶ cells. The induced yeast was spun down in table top centrifuge for 30 sec at max speed and the supernatant was discarded. The pellet was rinsed with PBS/BSA (phosphate buffered saline plus 0.1% BSA), centrifuged for 10 sec, and the supernatant discarded. The pellet was incubated with primary reagents. The desired concentration of biotinylated antigen and 1 μL 9e10 (1:100, Covance Laboratories, Inc., Madison, Wis.) were added to a final volume of 100 μL in PBS/BSA. The mixture was incubated at desired temperature for 30 min.

Larger volumes and longer incubation times were required for very low (<10 nM) antigen concentrations (see notes at end of protocol). The mixture was centrifuged, the supernatant was discarded, and the pellet rinsed with ice cold PBS/BSA. The mixture was again centrifuged and the supernatant was discarded from the rinse. The pellet was incubated on ice with secondary reagents. We added 97 μl ice cold PBS/BSA, 2 μl goat anti-mouse FITC conjugate (1:50, Sigma), and 1 μl streptavidin phycoerythrin conjugate (1:100, Molecular Probes). The mixture was incubated 30 min. The mixture was then centrifuged, the supernatant discarded, and the pellet rinsed with ice cold PBS/BSA. The mixture was again centrifuged and the supernatant discarded from the rinse. The cells were resuspended in 500 μl ice cold PBS/BSA and transferred to tubes for flow cytometry or FACS sorting.

An important consideration when labeling high affinity antibodies (<30 nM) was depletion of antigen from the labeling mixture. This resulted in a lower than expected concentration of soluble (free) antigen, and hence a lower signal. Sorting libraries under depletion conditions could reduce the difference in signal observed for improved clones compared to their wild-type counterparts. The equivalent concentration of yeast surface-displayed proteins when 0.2 OD₆₀₀-mL of yeast was added to a 100 μl volume was approximately 3 nM or less. To avoid depletion, we always used at least a 1 0-fold excess of antigen by adjusting the total volume and/or reducing the number of yeast added (as little as 0.05 OD₆₀₀-mL could be used).

Note that for labeling large libraries, it was advisable not to scale up directly. Instead we used 1 mL volume per 10⁸ cells labeled, keeping the reagent dilutions constant. Depletion could be especially severe with such high cell densities, however, and the experiment was designed to avoid such conditions.

Analyzing Clones and Libraries by Flow Cytometry

Once a yeast population is labeled, it was analyzed by flow cytometry. This allowed quantification of binding affinity by titrating antigen concentration. In addition to the samples to analyze, a negative control (no fluorophores), and two single positive controls Oust one fluorophore in each) were prepared. With standard filters installed, FITC was detected in the FL1 channel, while PE was detected by FL2 for the settings on most flow cytometers. However, some “bleed over” or spectral overlap was present in each channel, which needed to be compensated out. The negative control was used to set the voltage and gain on each of the detectors so that the negative population had order of magnitude intensity of one to ten. The single positive controls was used to adjust compensation so that no FITC signal was detected in FL2 and no PE signal in FL1.

In a titration, a gate was generally set on cells that expressed the antibody (i.e. FITC positive cells if the preceding labeling protocol was used) to eliminate non-expressing cells from quantification.

For sorting or analyzing a library, it was helpful to also prepare a labeled sample of the wild-type antibody and saturated library for comparison and to aid in drawing sort windows.

Sorting Yeast Surface Display Libraries by FACS

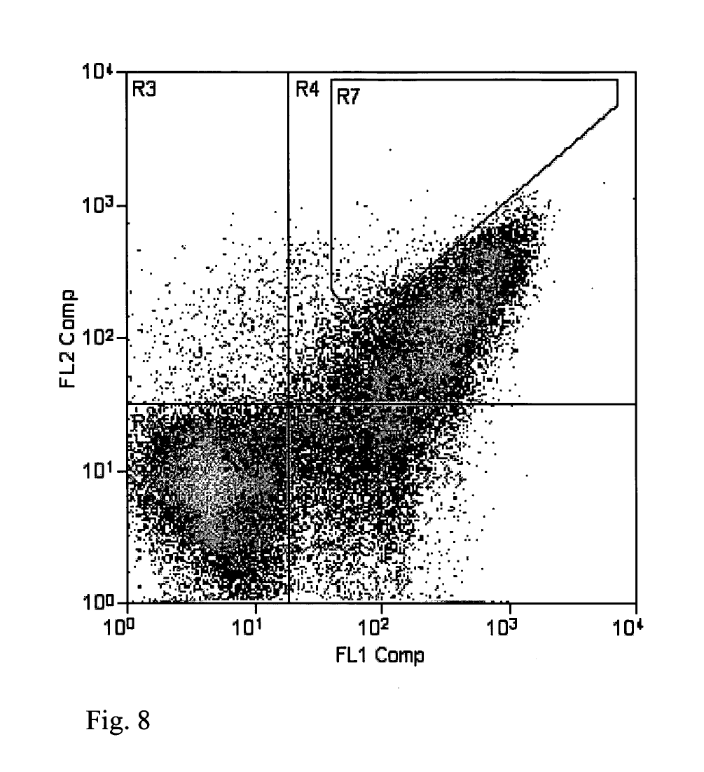

FACS is the most efficient and accurate way to sort yeast surface display libraries, although magnetic particle strategies have also been employed (Boder, E. T. et al., (1997) Nat Biotechnol 15, 553-557; Kieke, M. C. et al., (1997) Protein Eng 10, 1303-1310). To sort a library by FACS, we labeled cells according to the protocol above, taking into consideration the notes that follow the protocol. Equations describing the optimum labeling concentration for a first library sort are available (Boder, E. T. et al., (2000) Proc Natl Acad Sci U S A 97, 10701-10705), or we could simply choose a concentration that results in a weak signal (say, one fourth of the K_{d} value). We typically screened 10-100 times the number of independent clones that are in the library. When drawing a gate for collecting cells, it was advisable to use a window with a diagonal edge to normalize for expression, if a double positive diagonal was present (FIG. 8). If no diagonal was observed (little or no binding), the entire double positive quadrant was collected. Cells were sorted directly into SD+CAA with antibiotics such as penicillin and streptomycin to diminish the risk of bacterial contamination. Cells will grew to saturation in one (if>10⁵ cells are collected) or two (<10⁵) days. The very first time a library is sorted, gates were drawn conservatively (0.5% to 1% of the library is collected) to minimize the likelihood that an improved clone was missed. After the first sort, care was taken to note the number of cells collected, as this was the maximum number of independent clones remaining in the library. In subsequent sorts, when the library size had been reduced and the amount of sorting time necessary decreases, we brought several samples labeled under different conditions for sorting. These samples were sorted at increasing stringency to rapidly isolate the best clones. Sort gates covered the range of 0.01% of cells collected to 0.5%. All samples were analyzed and the one with the greatest improvement was chosen for further sorting. Typically the single best clone, or clones containing a consensus mutation, were isolated within 4 sorts.

The cells collected in the final sort were plated out for clonal analysis. The mutant plasmids could be recovered from yeast using the Zymoprep kit (Zymo Research, Orange, Calif.). The following primers were used for sequencing:

Forward Sequencing Primer: gttccagactacgctctgcagg (SEQ ID NO: 7)

Reverse Sequencing Primer: gattttgttacatctacactgttg (SEQ ID NO: 8)

Conclusion

The protocols and methods described here enabled engineering of scFv's by yeast surface display. The directed evolution process was often applied iteratively until the desired affinity is achieved. A single round of mutagenesis and screening typically resulted in 10- to 100-fold improvement in the Kd value, with largest improvements obtained when the wild-type affinity was low (say, low micromolar binding constant). A complete cycle of mutagenesis and screening, from wild-type clone to improved mutant clone, required conservatively approximately 3-6 weeks.

### Example 3

Background

Various anti-huntingtin antibodies are available include anti-huntingtin mAb 1C2, which decreases aggregation by 80% in filter assay (Heiser, V. et al., Proc Natl Acad Sci USA Jun. 6, 2000;97(12):6739-44); an anti-huntingtin scFv intrabody that reduced number of aggregates in cell model of HD (Messer, 2001); and an anti-polyproline scFv intrabody that reduced htt toxicity (Ko, J., et al., Brain Res Bull. Oct.-Nov. 1, 2001;56(3-4):319-29).

Drawbacks exist in the available antibodies and improvements we have made include: improved aggregation/toxicity inhibition properties; improved intracellular delivery of Abs with PTD's, and an improved ability to use the antibodies we produced to direct sub-cellular localization of Htt.

Antibody Library Construction

Antibody V gene cDNA from human peripheral blood lymphocytes, spleen, tonsil tissue was purchased commercially. The light and heavy chains isolated separately by PCR and ligated randomly to make scFv. The scFv DNA sequence was cloned into yeast surface display vector and transformed into yeast. The final diversity obtained was 1×10⁹.

Anti-Htt Antibodies Isolated from the Library

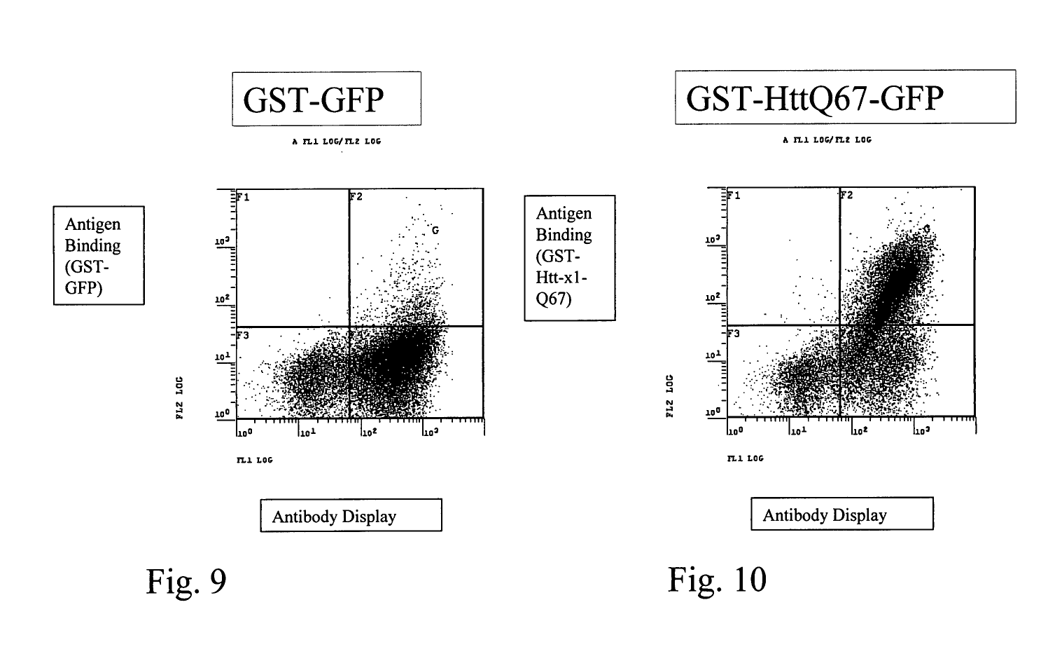

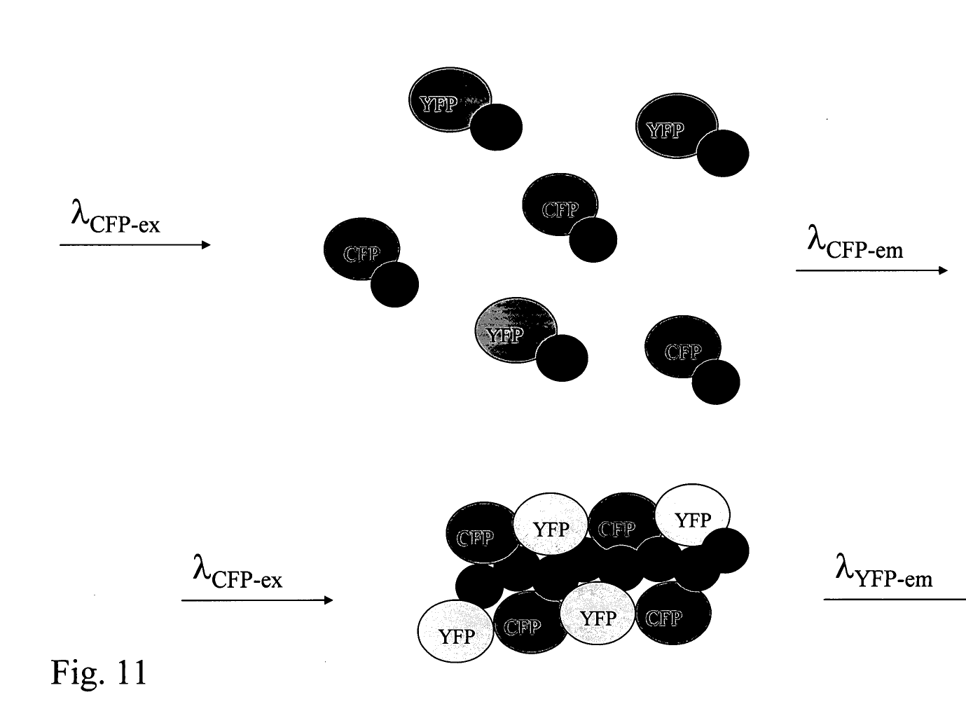

5×10⁸ cells from the library were incubated with 1 μM GST-Htt-x1-Q67-GFP and 9e10 (anti-c-myc mouse Mab). The cells were then rinsed and incubated on ice with PE labelled anti-GST and FITC labeled anti-mouse antibodies. The cells were then sorted by FACS for double positive cells. The positive cells were collected and grown several days, and the process that was repeated 3 times. FIGS. 9 and 10 illustrate antibodies isolated from the library (FIG. 9 is for antibody GST-GFP and FIG. 10 is for antibody GST-HttQ67-GFP). We utilized DNA fingerprinting with BstNI and identified 11 unique, viable clones.

Yeast HD FRET Model Constructs

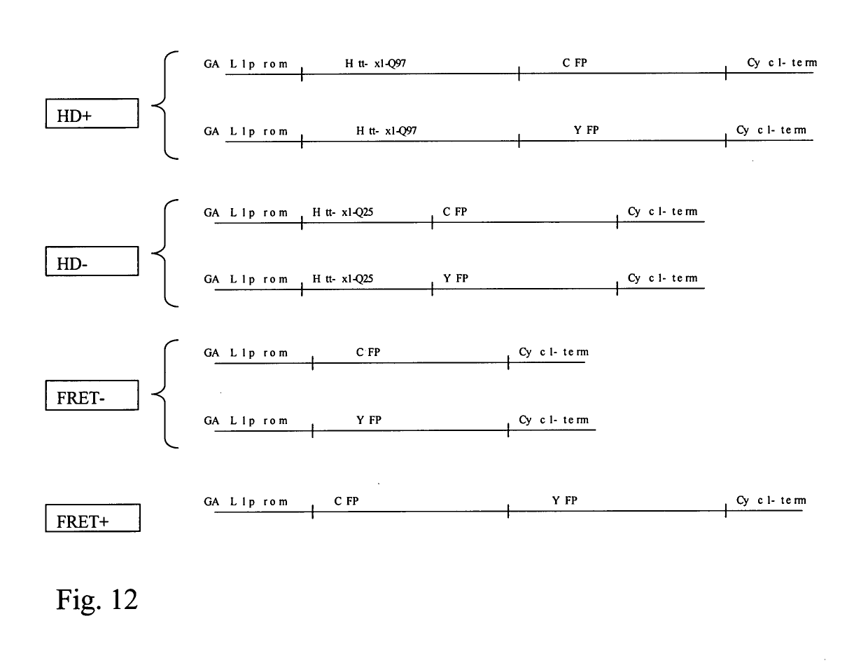

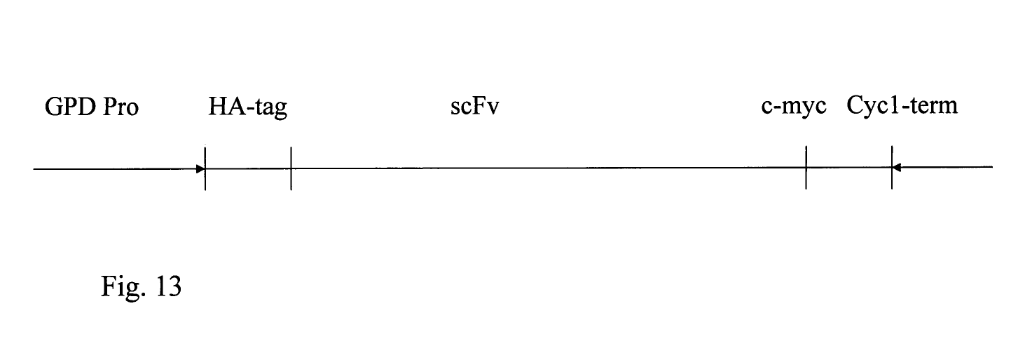

FIG. 11 shows a yeast HD FRET model. FIG. 12 shows yeast HD FRET model constructs. Bracketed constructs were co-expressed. For HD+ constructs co-expressed in yeast, a FRET signal was observed if CFP and YFP came into close contact (as in aggregation). HD− constructs served as a negative control to ensure that FRET signal was related to the expanded polyglutamine region, while FRET+ and FRET− controls are used to verify that FRET occurred at all. FRET was performed with Htt-x1-Q25 fused to YFP (red) and CFP (blue) and Htt-x1-Q97 fused to YFP (red) and CFP (blue) and demonstrated polyQ-length dependent aggregation of Htt.

Measuring FRET with Fluorescence Spectrophotometry

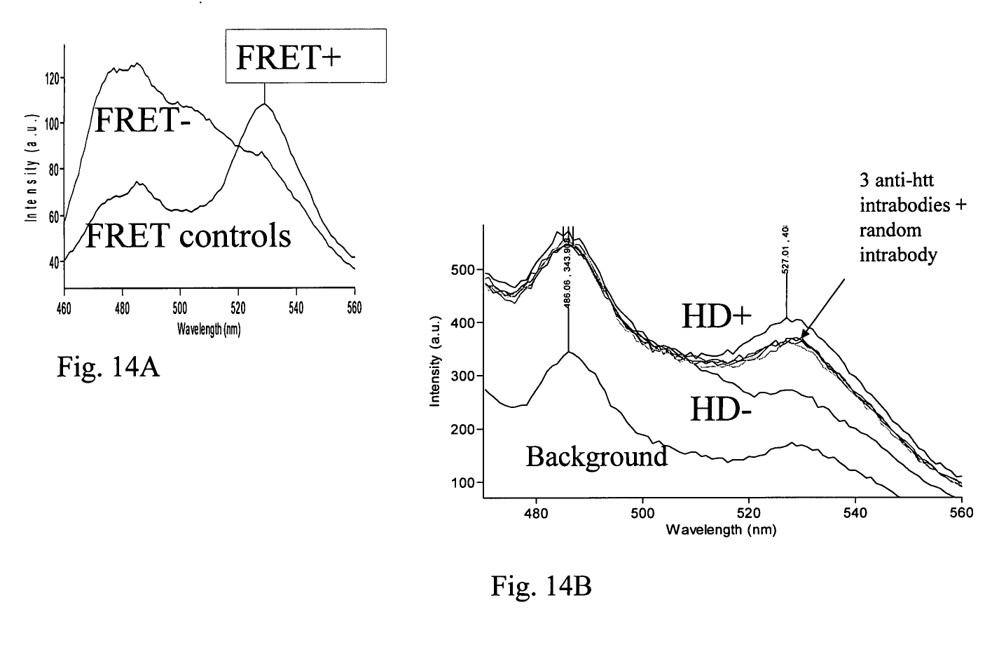

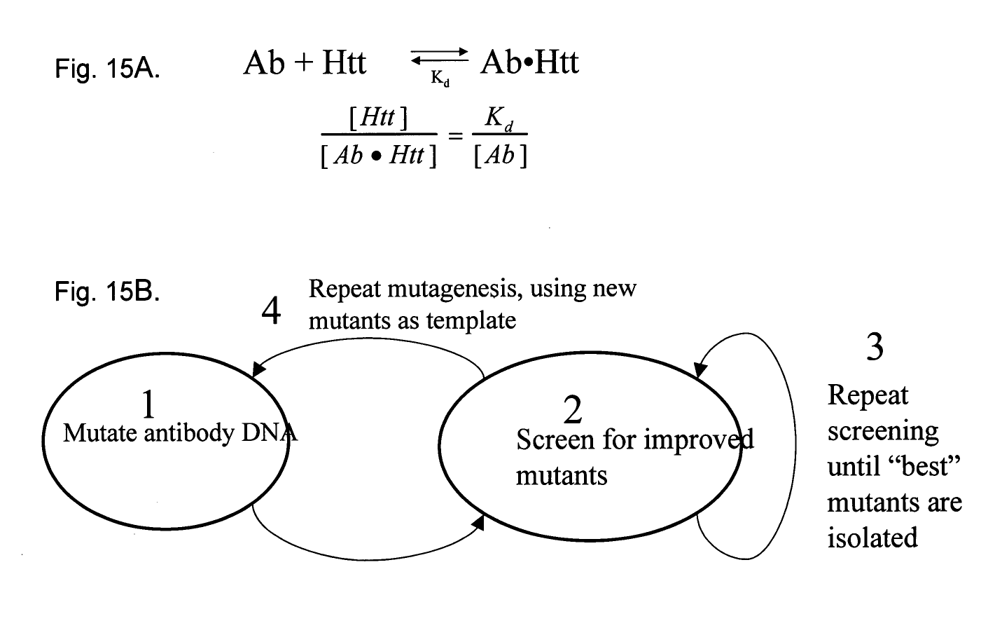

The antibodies were subcloned into cytoplasmic expression vectors (FIG. 13). Anti-htt scFv's were subcloned into a cytoplasmic expression vector (11 unique clones identified by DNA fingerprinting) and transformed into HD+ yeast. The negative control was from Ab selected at random from library. None of the anti-htt intrabodies reduce aggregation compared to control. The results were verified by quantitative fluorescence microscopy as illustrated in FIG. 14. The cell lines utilized included: HD+ (coexpress Htt-x1-Q97-CFP+Htt-x1-Q97-YFP), HD− (coexpress Htt-x1-Q25+Htt-x1-Q25-YFP), FRET+ (expresses CFP− YFP directly fused together), and FRET− (co-expresses CFP and YFP)

Measuring Aggregation in Cellular HD Models

Yeast and PC12 cells were transfected with inducible Htt-Q104-EGFP gene (obtained from Lindquist (yeast) and Housman (PC12) Labs). Aggregates begin to form in under 24 hours after induction. Fluorescence microscopy was used to generate images of cells with aggregates. The cells were PC 12 cells containing Htt-Q104-EGFP aggregate. The pixel value was proportional to intensity, which was a function of EGFP concentration. Aggregates had much higher EGFP concentration than soluble protein. The “Softworx” program (Applied Precision, Inc., Issaquah, Wash.) was used to set threshold and quantify aggregation.

Directed Evolution

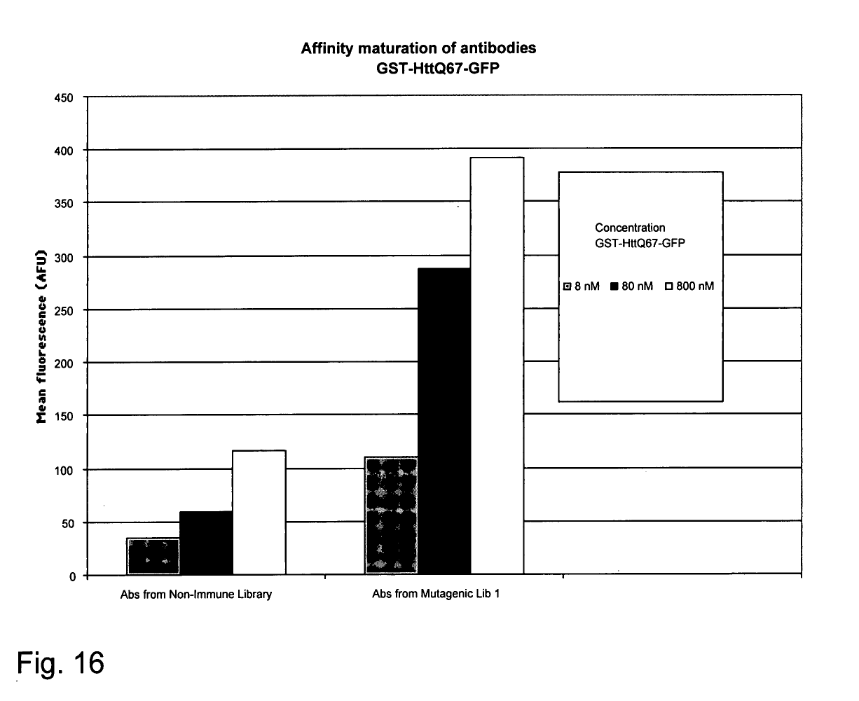

FIG. 15 illustrates methods of directed evolution of the antibodies. In the directed evolution method we mutated antibody DNA, screened for improved mutants, repeated screening until “best” mutants are isolated, and repeated the mutagenesis, using new mutants as template.

Affinity Engineering

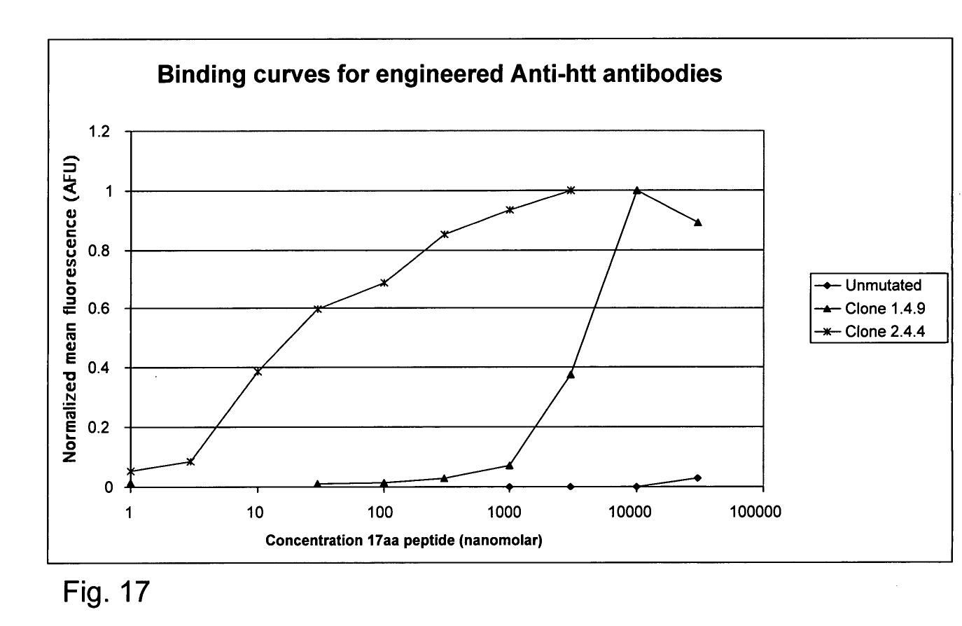

Higher affinity antibodies were sought. Abs bound multivalent antigen at 1 μM and we anticipated that monovalent affinity could be much lower. A mixture of 11+ clones were used as template for error-prone PCR using nucleotide analogs to generate mutants. The library consisted of 6×10⁶ transformants. The library was sorted four times against first 20 amino acids of Htt biotinylated peptide at 1 μM. FIG. 16 illustrates the results of affinity maturation of antibodies GST-HttQ67-GFP and shows antibodies from non-immune library and antibodies from our mutagenic library.

Second Round of Affinity Engineering

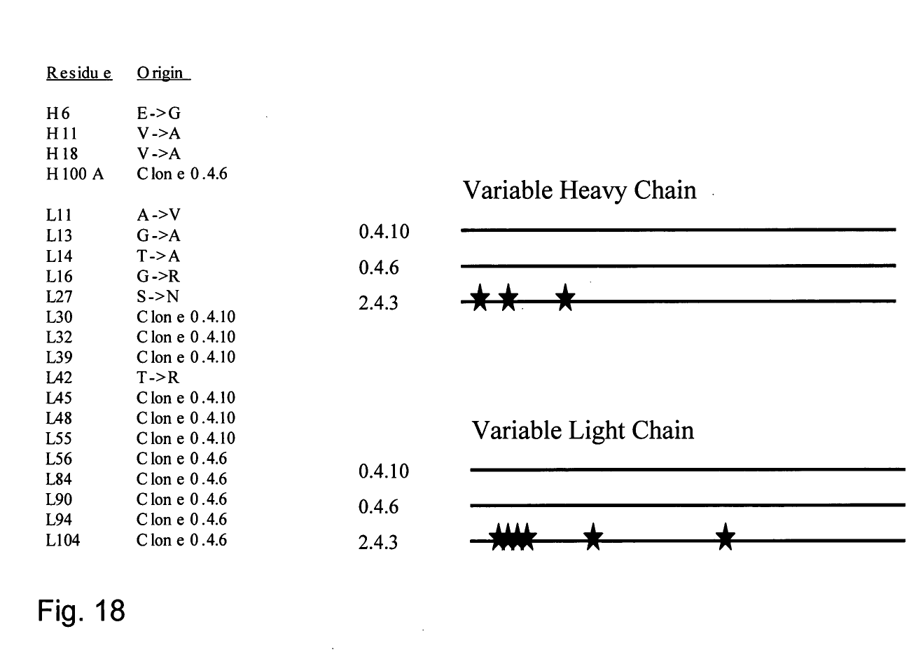

The mixture of final sort from first mutagenic library (80%), final sort from non-immune library (10%), and unsorted non-immune library (10%) was used as template. Nucleotide analogue PCR was used to generate mutants. The mutants were transformed into yeast (˜2×10⁶ mutants). The mutants were sorted four times against 100 nM peptide. FIG. 17 illustrates that the antibody affinity improved over 5000-fold after two rounds of mutagenesis and screening.

Sequencing Results

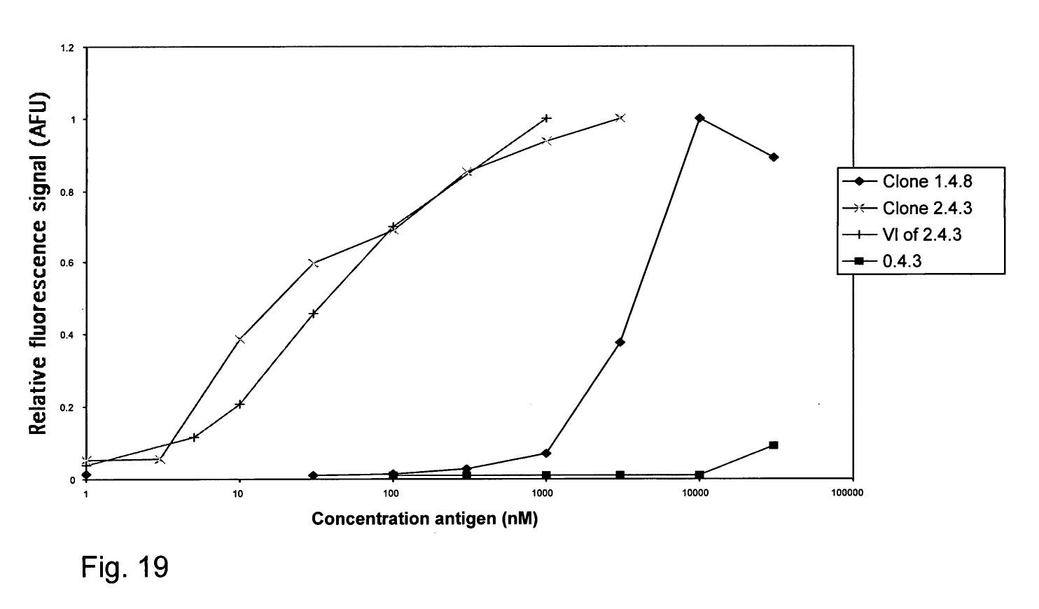

The results indicated that Clone 2.4.3 was derived from 0.4.8, and had 9 amino acid mutations. Also the results indicated that no clone from 1.4.x was in the second enriched library. In addition, the results showed that the clone has one unusual mutation in framework residues (<1% of Kabat database). Results indicated that the best clone acquired mutations through both DNA shuffling and error-prone PCR (see FIG. 18).

Light Chain Only Binds Htt

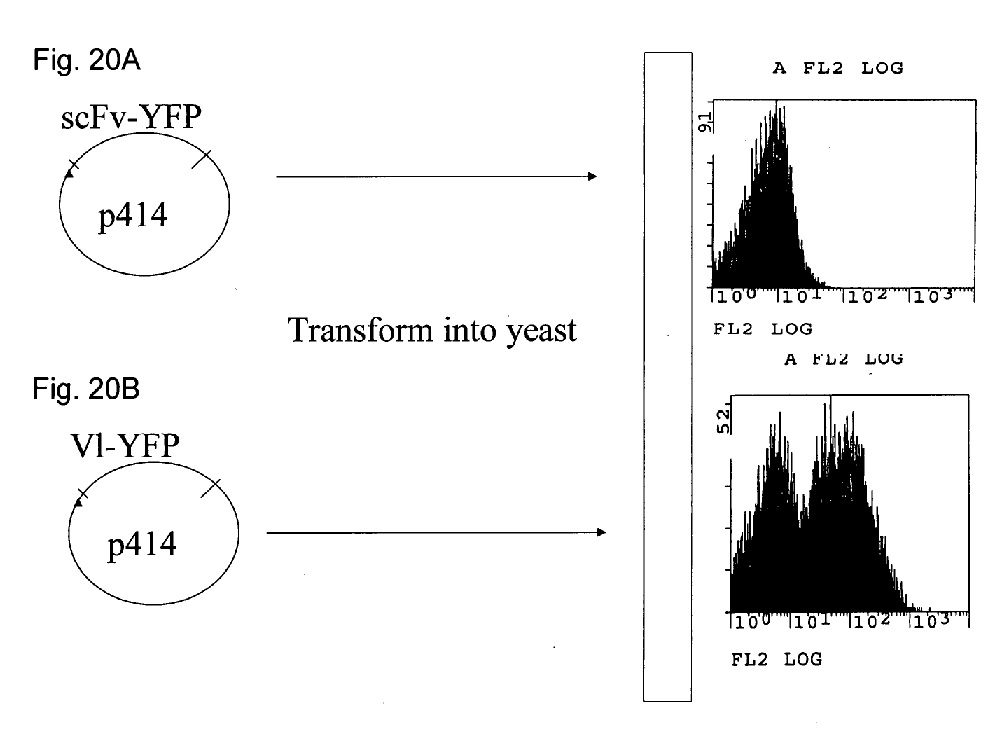

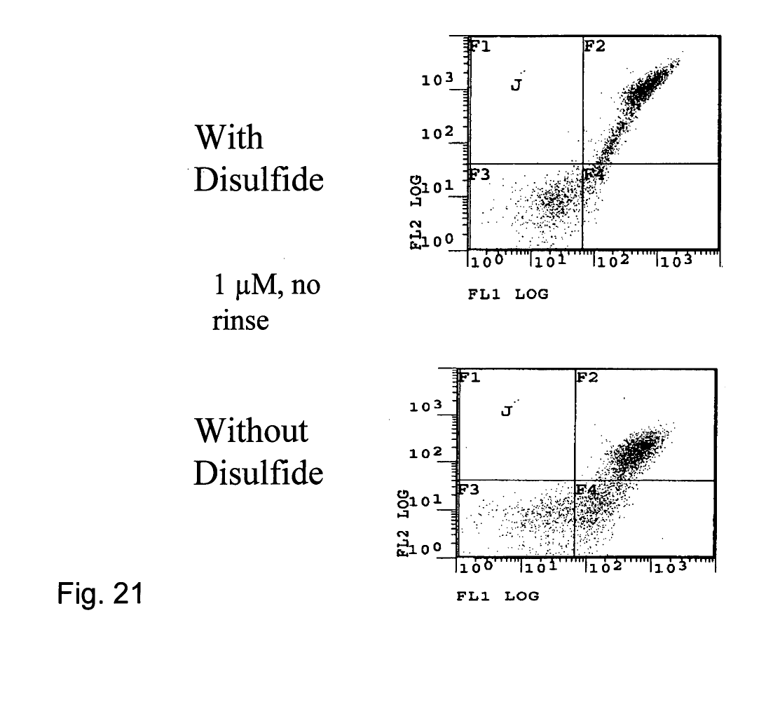

A third mutagenic library was made experimenting with new mutagenic conditions. No higher affinity mutants were obtained, but one mutant was 2.5x better expressed and retained the same affinity (30-50 nM). The improved mutant was the light chain only of scFv. FIG. 19 is a graph of results indicating that VL domain of 2.4.3 retains its binding activity. FIG. 20 illustrates that the single domain antibody is well expressed in cytoplasm as a YFP fusion. FIG. 1 illustrates that the single-domain Ab inhibits Htt aggregation.

Disulfide Knock-Out Binds Weakly

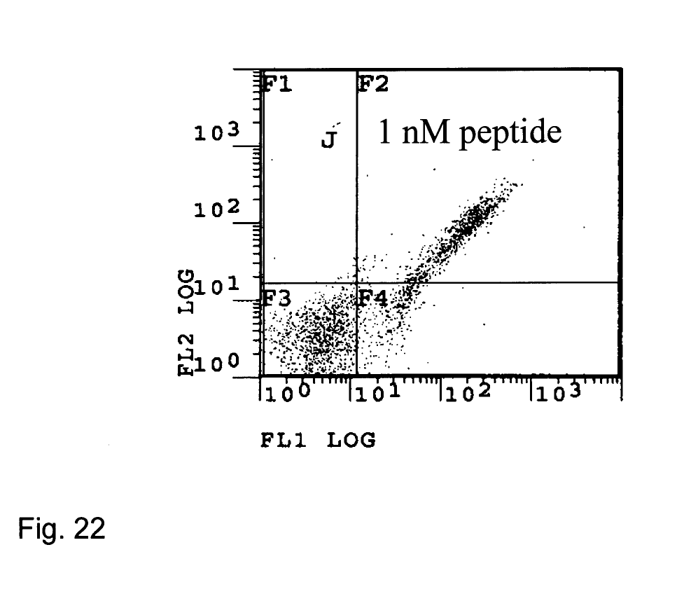

It was determined that knocking out disulfide bond ablates binding (30-50 nM drops to low micromolar affinity, cysteines were mutated to valine or alanine). This effect is demonstrated in FIG. 21, which shows binding for antibodies with and without the disulfide bond. The disulfide-free antibody was then affinity matured. Three rounds of mutation and screening restored binding to ˜10 nM, by 7 amino acid substitutions (FIG. 22).

Conclusion

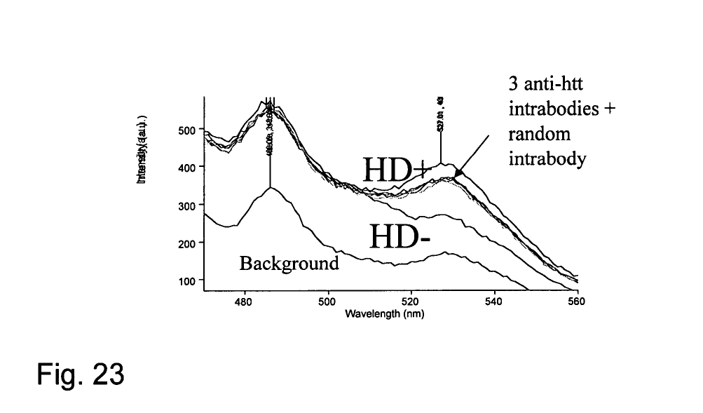

We have demonstrated methods through which an anti-htt single-chain antibodies isolated from a non-immune human library that are ineffective at blocking cellular htt aggregation, can be affinity matured to ˜30 nM. We also have discovered that light chain was responsible for binding and that VL only had superior intracellular expression. Our results show that VL eliminated htt aggregation in a cell-free assay and that knocking out disulfide bond ablated binding. We then were able engineer a disulfide-free VL to an affinity of ˜10 nM. FIG. 23 demonstrates a test-engineered antibody for aggregation inhibition and demonstrates binding of three anti-htt antibodies.

### Example 4

The Engineered antibody described in Example 3 is used to direct Htt localization. The roles of aggregation and localization in HD are investigated. Peptide transduction domains (PTDs) are linked to single-domain antibodies and used to deliver single-domain antibodies inside cells.

### Example 5

Potent Inhibition of Huntingtin Aggregation and Cytotoxicity by a Disulfide Bond-Free Single Domain Intrabody

Introduction

Huntington's Disease (HD) is a progressive neurodegenerative disorder caused by an expansion in the number of polyglutamine-encoding CAG repeats in the gene that encodes the huntingtin (htt) protein. A property of the mutant protein that is intimately involved in the development of the disease is the propensity of the glutamine-expanded protein to misfold and generate an N-terminal proteolytic htt fragment that is toxic and prone to aggregation. Intracellular antibodies (intrabodies) against htt have been shown to reduce htt aggregation by binding to the toxic fragment and inactivating it or preventing its misfolding. Intrabodies may therefore be a useful gene therapy approach to treatment of the disease. However, high levels of intrabody expression have been required to obtain even limited reductions in aggregation. We have engineered a single domain intracellular antibody against huntingtin for robust aggregation inhibition at low expression levels, by increasing its affinity in the absence of a disulfide bond. Further, the engineered intrabody V_{L}12.3, rescued toxicity in a neuronal model of HD. We also found that V_{L}12.3 inhibited aggregation and toxicity in a S. cerevisiae model of HD. V_{L}12.3 is significantly more potent than earlier anti-htt intrabodies, and is a potential candidate for gene therapy treatment for HD. This method was developed to improve affinity in the absence of a disulfide bond in order to improve intrabody function. The demonstrated importance of disulfide bond-independent binding for intrabody potency allows a generally applicable approach to the development of effective intrabodies against other intracellular targets.

In Huntington's disease, a proteolytic fragment of the huntingtin protein that contains an expanded polyglutamine stretch misfolds and forms beta-sheet rich aggregates. Intracellularly expressed antibodies with specificity for huntingtin have been shown to reduce aggregation and toxicity in cellular and organotypic slice culture models of HD (Colby, D. W. et al., J Mol Biol 342: 901-912, 2004; Khoshnan, A., et al., Proc Natl Acad Sci U S A 99: 1002-1007, 2002; Lecerf, J. M. et al., Proc Natl Acad Sci U S A 98: 4764-4769, 2001; Murphy, R. C. et al., Brain Res Mol Brain Res 121: 141-145, 2004). However, high intrabody expression levels have been required to obtain moderate reductions in aggregation and toxicity. This has proven to be a barrier to the development of a treatment for HD with intracellular antibodies via gene therapy, given the limited ability of viral vectors to deliver genes to the CNS. Intrabodies are an attractive means of manipulating intracellular protein function. However, their success has been limited largely to use in target validation, rather than experimental therapy in preclinical disease models, in part due to their limited efficacy. A key problem arises from the conditions under which antibodies against intracellular targets are isolated and engineered. With the exception of the yeast two-hybrid approach to intrabody isolation (Visintin, M. et al., Proc Natl Acad Sci U S A 96: 11723-11728, 1999), antibodies are isolated and engineered under oxidizing conditions by yeast or phage display (Colby, D. W. et al., J Mol Biol 342: 901-912, 2004; Emadi, S. et al., Biochemistry 43: 2871-2878, 2004; Gennari, F. et al., J Mol Biol 335: 193-207, 2004), where stabilizing disulfide bonds form; however, disulfide bonds do not form as readily in the reducing environment of the cytoplasm, where intrabodies are intended to function. Lead optimization or incremental improvement of intrabody function has not been reported to date with a yeast two-hybrid approach, perhaps due to the qualitative nature of that screening system.

Previously, we reported the isolation of a single-chain antibody (scFv) specific for the first 20 amino acids of huntingtin, and its reduction to a single variable light chain (V_{L}) domain, in order to enable intracellular expression and mild inhibition of htt aggregation (Colby, D. W. et al., J Mol Biol 342: 901-912, 2004). We have now engineered this V_{L} intrabody for robust and effective inhibition of aggregation and cytotoxicity by removing the disulfide bond to make intrabody properties independent of redox environment, whether intracellular or extracellular. First, the cysteines that form the disulfide bond were mutated to hydrophobic residues, a technique shown to be effective for obtaining higher yields of active antibody expressed from E. coli (Proba, K. et al., J Mol Biol 275: 245-253, 1998). This resulted in an unexpectedly large decrease in the intrabody's affinity for its antigen. Iterative rounds of mutation and screening were then applied to improve the intrabody's affinity, a process that mimics affinity maturation in the immune system. We found that the ability to block htt exon I aggregation correlated with antigen binding affinity in the absence of disulfide bonds. Disulfide-independent binding affinity and intracellular antibody expression levels (Colby, D. W. et al., J Mol Biol 342: 901-912, 2004; Rajpal, A. et al., J Biol Chem 276: 33139-33146, 2001; Arafat, W. et al., Cancer Gene Ther 7: 1250-1256, 2000; Zhu, Q. et al., J Immunol Methods 231: 207-222, 1999), appear to be the two important design variables for the development of highly functional intracellular antibodies.

Yeast surface display [YSD, (Boder, E. T. et al., Nat Biotechnol 15: 553-557, 1997)] is a technique for isolation of novel antibodies (Feldhaus, M. J. et al., Nat Biotechnol 21: 163-170, 2003), improving protein function (Colby, D. W. et al., Methods Enzymol 388: 348-358, 2004; Graff, C. P. et al., Protein Eng Des Sel 17: 293-304, 2004; Rao, B. M. et al., Protein Eng 16: 1081-1087, 2003; Boder, E. T. et al., Proc Natl Acad Sci U S A 97: 10701-10705, 2000), and analysis of protein properties (Colby, D. W. et al., J Mol Biol 342: 901-912, 2004, Cochran, J. R. et al., J Immunol Methods 287: 147-158, 2004; Orr, B. A. et al., Biotechnol Prog 19: 631-638, 2003; Shusta, E. V. et al., J Mol Biol 292: 949-956, 1999). In this system, the gene for a protein of interest is fused to the gene for the yeast mating protein (Aga2p) and to epitope tags, such as c-myc, for detection. When transformed into an appropriate yeast strain, the protein is displayed on the yeast cell wall, where it is accessible to antigens or other interaction partners and immunofluorescent reagents in solution. In this way, the properties of individual proteins may be analyzed by flow cytometry, or libraries of expressed proteins may be sorted to isolate clones with desired properties by fluorescence activated cell sorting (FACS). We have used this technique to engineer an intrabody for high affinity without a disulfide bond, allowing facile transfer of this property to the intracellularly expressed intrabody.

Methods

Yeast surface display. The cysteine residues of yeast displayed V_{L} (1) were changed to valine and alanine (C22V, C89A) by site directed mutagenesis of the V_{L} gene using QuikChange PCR (Stratagene, La Jolla, Calif.). Yeast surface display labeling experiments to measure expression and binding were conducted as previously described (Boder, E. T. et al., Methods Enzymol 328: 430-444, 2000). A peptide consisting of the first 20 amino acids of htt was used as the antigen (MATLEKLMKAFESLKSFQQQ-biotin (SEQ ID NO:9), synthesized by the MIT biopolymers lab). The antigen was synthesized to contain three glutamines because the beginning of the polyglutamine region would be an ideal target for interfering with the misfolding of htt exon I. Affinity maturation of V_{L},C22V,C89A relied upon protocols previously described (Colby, D. W. et al., Methods Enzymol 388: 348-358, 2004). Briefly, the V_{L},C22V,C89A gene was used as the template for the creation of a library of point mutants through error-prone PCR using nucleotide analogues. The resulting PCR products were amplified and transformed into yeast along with digested pCTCON (a yeast surface display vector) to create a library through homologous recombination (Raymond, C. K. et al., Biotechniques 26: 134-138, 140-141, 1999). The library had a diversity of 3×10⁷ intrabody mutants displayed on the surface of yeast. This library was sorted 4 times by FACS to isolate mutants with approximately 10-fold improvement in affinity, as measured by titration with the 20 amino acid htt peptide. These mutants were then used as the template in the next round of library generation. The entire process, from library generation to isolation of improved mutants, was repeated three times to yield V_{L}12.3. FACS sorting was performed using a Cytomation Moflo FACS machine by the staff of the MIT Flow cytometry core facility. All constructs and clones were sequenced at the MIT biopolymers lab.

Mammalian cell culture, aggregation assay, and toxicity assays. ST14A cells (Cattaneo, E. et al., J Neurosci Res 53: 223-234, 1998), HEK293, and SH-SY5Y cells were cultured according to standard protocols. (The ST14A cell line was generously provided by E. Cattaneo from the University of Milan, Milan, Italy). C-terminal his6 tagged intrabody constructs were expressed from a pcDNA3.1 vector under the control of a CMV promoter. The method used to quantify the effect of intrabodies on intracellular htt exon I aggregation in the three cell lines mentioned above is described in detail elsewhere (Colby, D. W. et al., J Mol Biol 342: 901-912, 2004); briefly, cells were transiently transfected using lipofectamine (Invitrogen) or similar reagents and presence of aggregates was monitored by fluorescence microscopy. Transfection efficiencies and expression levels of httex1Q97-GFP were monitored by flow cytometry on a Moflo FACS machine (Cytomation, Ft. Collins, Colo.). Cell lysis, preparation of Triton soluble lysates and immunoblots were carried out as described (Webster, J. M. et al., J Biol Chem 278: 38238-38246, 2003). Triton insoluble fractions were prepared by resuspending the Triton X-100 insoluble pellet in water, followed by sonication and centrifugation at 16,000 g for 10 min.; the final pellet was resuspended in SDS gel loading buffer before processing in immunoblots with a monoclonal anti-htt recognizing the first 17 amino acids of the htt protein (m445).

Intracellular expression levels of intrabodies were measured by anti-His (antibody from Santa Cruz Biotechnology, Santa Cruz, Calif.) western blot.

For the MTT (3-(4,5-dimethylthiazol-2-yl)-2,5-diphenyltetrazolium bromide) assay, which was used to measure metabolic activity, transiently transfected ST14A cells were sorted based on GFP signal to collect populations expressing GFP or httex1-GFP transgenes. These cells were sorted directly into 96-well plates, 35,000 cells per well in 100 ml maintenance media. The MTT assay (kit from ATCC, Manassas, Va.) was then performed according to the manufacturer's protocol. A Fluorostar Optima96-well plate reader (BMG Labtechnologies, Offenburg, Germany) was used to measure absorbance of the metabolic product at 570 nM.

Yeast cell culture, aggregation, and toxicity. Yeast media was prepared based on standard protocols (1991 Methods in Enzymology) using complete supplemental mixtures (BIO101, Qbiogene, Irvine, Calif.). Transformation of yeast was performed as described previously (Ito, H. et al., J Bacteriol 153: 163-168, 1983). Yeast integrating plasmids containing galactose inducible promoters (pRS303 backbone (Sikorski, R. S. & Hieter, P. Genetics 122: 19-27, 1989)) for the expression of huntingtin exon-fragments were linearized by digestion with BstXI prior to transformation. V_{L}12.3-YFP was subcloned into p414 (ATCC), which also contains a galactose inducible promoter. Filter retardation assays of aggregated material were done essentially as described previously (Muchowski, P. J. et al., Proc Natl Acad Sci U S A 97: 7841-7846, 2000). For the induction of expression of the huntingtin fragment in yeast, cultures were grown at 30° C. in raffinose-containing liquid media and transferred to galactose-containing media. In order to measure growth, yeast cells were diluted to a final OD 600 nm of 0.05 and transferred to a microtiter plate. Yeast cultures were grown at 30° C. with intermittent, intensive shaking on the Bioscreen C (Growth Curves USA, Piscataway, N.J.) for 48 hrs with OD measurements taken every 2 hours. Western blot analysis of V_{L}12.3-YFP and httex1Q72-CFP with anti-GFP antibodies indicated that 8 the intrabody was present at lower protein concentrations than the htt exon I fragment.

Results

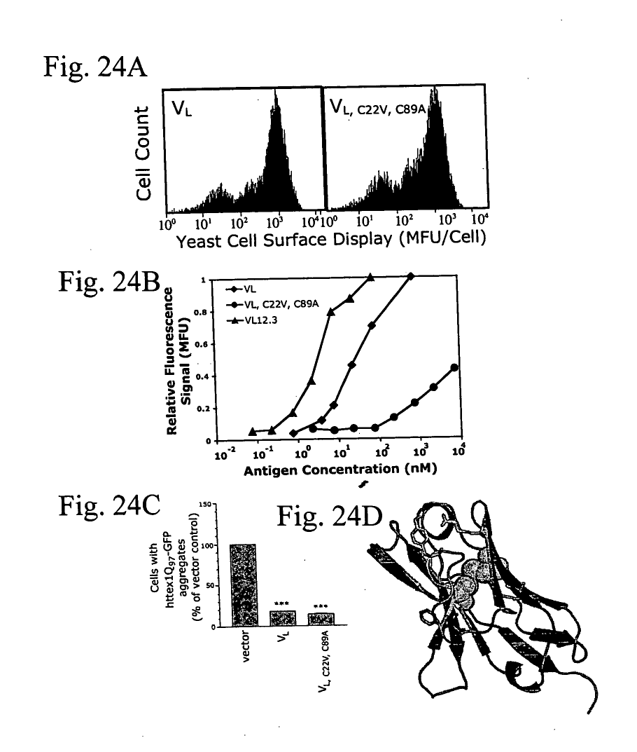

Elimination of anti-htt V_{L} intrabody's disulfide bond reduces affinity for huntingtin. Intracellular expression of antibody fragments leads to incomplete formation of structurally important disulfide bonds. To determine the impact of incomplete disulfide bond formation on V_{L} expression and affinity for huntingtin, the cysteines of yeast surface-displayed V_{L} were mutated to valine and alanine (C22V, C89A) (Proba, K. et al., J Mol Biol 275: 245-253, 1998), to make mutant V_{L}, C22V, C89A. Yeast cell surface protein expression levels, which can be monitored by the presence of a C-terminal c-myc tag detected by immunofluorescence and flow cytometry, have been shown to correlate strongly with protein stability (Orr, B. A. et al., Biotechnol Prog 19: 631-638, 2003; Shusta, E. V. et al., J Mol Biol 292: 949-956, 1999). Significantly, yeast cell surface expression levels of V_{L}, C22V, C89A were comparable to those of V_{L}, suggesting that the absence of the disulfide bond did not significantly alter stability of the protein (FIG. 24A). A negative peak can be seen just above a fluorescence value of 101, due to cells that have lost the expression plasmid.

We then measured the affinity of the wild type V_{L} and mutant V_{L}, C22V, C89A for a biotinylated peptide antigen consisting of the first 20 amino acids of htt, by titration of the yeast surface displayed intrabodies (FIG. 24B, diamonds and circles, respectively). The mutant lacking a disulfide bond exhibited a binding affinity 2-3 orders of magnitude lower than the wild type intrabody (approximate affinities are V_{L} ˜30 nM, V_{L}, C22V, C89A>10 μM), indicating the importance of disulfide bond formation in maintaining the structural integrity of the antigen binding site of the intrabody. Since disulfide bonds are not thermodynamically favored in the reducing environment of the cytoplasm, the intracellular affinity of V_{L} is expected to be on the order of that of the mutant lacking the disulfide bond.

Elimination of disulfide bond does not affect aggregation inhibition properties of intrabody in transiently transfected mammalian cell model of HD. To ensure that mutation of the cysteine residues that form the disulfide bond of the yeast surface displayed V_{L} mimics intracellular expression, we measured the effect of disulfide bond elimination on the ability of the intrabody to block htt aggregation when transiently transfected into mammalian cells at a high plasmid ratio relative to htt. ST14A cells were co-transfected with httex1Q97-GFP (also in pcDNA3.1) and either an empty vector, V_{L}, or V_{L}, C22V, C89A, at a 2:1 intrabody:htt plasmid ratio. Twenty-four hours posttransfection, cells with aggregates were counted. Both the wild-type intrabody and the mutant lacking cysteines inhibited aggregation to the same extent (FIG. 24C), when expressed at high levels. The equivalent aggregation inhibition of V_{L} and V_{L}, C22V, C89A, despite the almost 1,000-fold difference in affinities of the intrabodies under oxidizing extracellular expression conditions, strongly suggests that the disulfide bond in V_{L} does not form in the cytoplasm.

Intrabody lacking disulflde bond engineered for high affinity by directed evolution. Since the intracellular affinity of the V_{L} was relatively low, we hypothesized that more potent aggregation inhibition could be achieved by engineering V_{L}, C22V, C89A for higher affinity. Random mutagenesis of the V_{L}, C22V, C89A gene was carried out using error-prone PCR. The resulting PCR fragments were transformed into yeast along with a yeast surface display vector to create a library through homologous recombination (Raymond, C. K. et al., Biotechniques 26: 134-138, 140-141, 1999). This library had a diversity of approximately 3×10⁷ intrabody mutants displayed on the surface of yeast. Iterative rounds of FACS sorting were used to isolate new mutants with improved affinity. The process of mutagenesis and sorting resulted in an approximately 10-fold improvement in binding affinity. The improved mutants obtained were used as the template for the next round of library creation; the entire mutagenesis and sorting process was repeated three times. After the third round, one mutant designated V_{L}12.3 was identified with significantly improved affinity, (titration shown in FIG. 24B; approximate Kd˜5 nM). The amino acid sequence of V_{L}12.3 is set forth as SEQ ID NO:10:

MGSQPVLTQSPSVSAAPRQRVTISVSGSNSNIGSNTVNWIQQLPGRAPEL
LMYDDDLLAPGVSDRFSGSRSGTSASLTISGLQSEDEADYYAATWDDSLN
GWVFGGGTKVTVLSGHHHHHH.

The improved mutant was sequenced and found to have gained 4 mutations (F37I, Y51D, K67R, A75T); continued absence of the cysteine residues was also confirmed. Three of the four mutations were in framework positions (residues in antibody variable domains that do not generally form contacts with antigens); only one was in a complementarity determining region (Y51D in CDR L2). The locations of the mutations are included in a homology model (FIG. 24D; homology model generated at Web Antibody Modeling (antibody.bath.ac.uk/index.html).

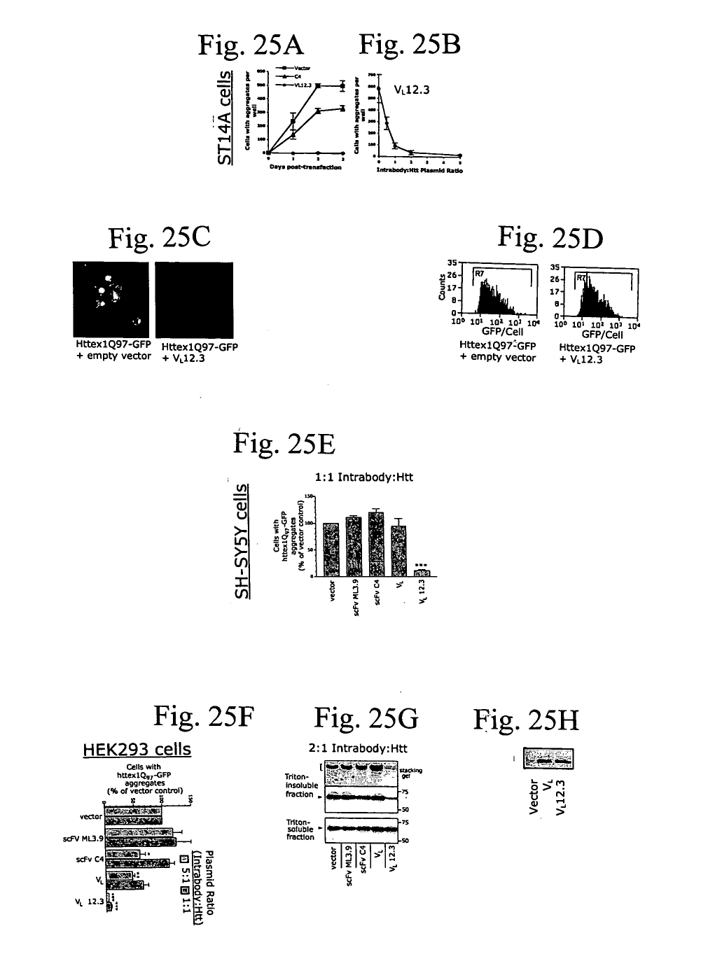

Engineered intrabody V_{L}12.3 robustly blocks aggregation in transiently transfected mammalian cell models of HD. To determine whether V_{L}12.3 has improved huntingtin aggregation inhibition properties, various cell lines were transiently co-transfected with httex1Q97-GFP and V_{L}12.3, and the formation of aggregates was monitored by fluorescence microscopy and western blotting. In some experiments, an intrabody that lacked specificity for huntingtin (ML3-9) and an empty control vector were tested as a negative controls, and previously reported C4 (Lecerf, J. M. et al., Proc Natl Acad Sci U S A 98: 4764-4769, 2001) and V_{L} (Colby, D. W. et al., J Mol Biol 342: 901-912, 2004) were included for comparison. First, experiments were performed using intrabody to htt plasmid ratios of 5: 1. In previous work (Colby, D. W. et al., J Mol Biol 342: 901-912, 2004; Khoshnan, A., et al., Proc Natl Acad Sci U S A 99: 1002-1007, 2002; Lecerf, J. M. et al., Proc Natl Acad Sci U S A 98: 4764-4769, 2001), such high levels of intrabody overexpression were required to accomplish moderate reduction of aggregate formation. V_{L}12.3 exhibited the ability to essentially ablate aggregation at these high levels of expression, as shown in FIG. 25A (circles), for V_{L}12.3 in ST14A cells, compared to C4 (triangles) and empty vector (squares). Significantly, aggregation inhibition persisted over a period of several days.

Given the strong capability of V_{L}12.3 to reduce the formation of aggregates at high expression levels, we then studied the dose response of aggregate formation by varying the ratio of intrabody to htt plasmid. As shown in FIG. 25B, V_{L}12.3 blocked aggregation significantly even when expressed at very low levels (0.5:1 intrabody:htt plasmid ratio). The formation of aggregates was reduced by nearly 80% when the intrabody plasmid was present in a 1:1 ratio with htt plasmid, and greater than 90% when present at higher levels. Sample images with and without V_{L}12.3 are shown in FIG. 25C.

Flow cytometry was used to determine whether expression levels of httex1Q97-GFP were different in the presence of the intrabody; expression levels were comparable for samples with intrabody compared to empty vector (FIG. 25D). Therefore, the decrease in aggregation did not occur simply as a result of inhibiting httex1Q97-GFP expression. Efficacy of V_{L}12.3 was characterized and compared to two previously described intrabodies (C4 and V_{L}) in other cell lines, both by fluorescence microscopy and western blotting analysis. In SH-SY5Y human neuroblastoma cells at a 1:1 intrabody:htt ratio only V_{L}12.3, and not earlier intrabodies, effectively reduced aggregation (FIG. 25E). Aggregation inhibition properties of V_{L}12.3 in HEK293 cells (FIG. 25F) were comparable to those observed in ST14A and SH-SY5Y cells. Partial dose-response curves are shown for each intrabody. Especially noteworthy is the ability of V_{L}12.3 to inhibit aggregation when used at a plasmid ratio (1:1 intrabody to htt) which was completely ineffective with previously reported intrabodies.

While microscopy confirmed that fewer cells contain visible aggregates when cotransfected with V_{L}12.3, we also sought to confirm a reduction in total aggregated htt protein. Western blotting analysis of Triton-soluble and Triton-insoluble htt fractions was performed on cell lysates obtained from HEK293 cells (FIG. 25G), transiently transfected using a 2:1 ratio of intrabody:htt plasmid. Significantly reduced levels of aggregated material were detected in the Triton-insoluble fractions for cells cotransfected with V_{L}12.3 and httex1Q97-GFP, while co-transfection of httex1Q97-GFP with any of the other intrabodies resulted in amounts of aggregated material comparable to negative control. Coexpression of intrabodies did not decrease the amount of material in the Triton-soluble fraction.

V_{L} was expressed at levels equivalent to or slightly higher than V_{L}12.3, as measured by anti-His6 western blot (FIG. 25H).

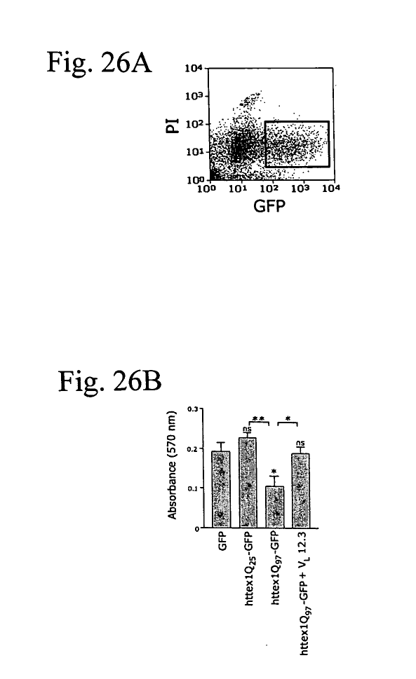

Engineered V_{L}12.3 inhibits toxicity in neuronal cell culture model of HD. Energy metabolism impairment and mitochondrial dysfunction have been described in cellular models of HD as well as in HD patients (Choo, Y. S. et al., Hum Mol Genet 13: 1407-1420, 2004; Leenders, K. L. et al., Mov Disord 1: 69-77, 1986). To see if the engineered V_{L}12.3 intrabody could reduce toxicity in mammalian cells in addition to blocking aggregation, the MTT (3-(4,5-dimethylthiazol-2-yl)-2,5-diphenyltetrazolium bromide) assay was used to measure the mitochondrial activity of transiently transfected ST14A cells (Mosmann, T., J Immunol Methods 65: 55-63, 1983). ST14A cells were transfected with either GFP, httex1Q25-GFP, or httex1Q97-GFP. Forty-eight hours post-transfection, live GFP positive cells were sorted by FACS. The ability of the cells to metabolize MTT during four additional hours of culture was measured. Compared to cells expressing GFP or httex1Q25-GFP, cells expressing httex1Q97-GFP exhibit an attenuated ability to reduce MTT (FIG. 26). Co-transfection with V_{L}12.3 at a 2:1 ratio resulted in completely restored ability to metabolize MTT, indicating normal levels of mitochondrial activity.

Engineered V_{L}12.3 blocks aggregation and cytotoxicity in a yeast model of HD. S. Cerevisiae is likely the simplest in vivo model of HD, exhibiting both huntingtin aggregation and cytotoxicity (Krobitsch, S. et al., Proc Natl Acad Sci U S A 97: 1589-1594, 2000; Meriin, A. B. et al., J Cell Biol 157: 997-1004, 2002). To determine whether the engineered intrabody could prevent these HD phenotypes in yeast, S. cerevisiae strains expressing both a huntingtin exon I protein (with either Q25 or Q72) fused to cyan fluorescent protein (httex1Q25-CFP and httex1Q72-CFP) and a V_{L}12.3-yellow fluorescent protein fusion (V_{L}12.3-YFP) on galactose-inducible promoters were made. Negative control strains were also constructed with an empty vector in place of V_{L}12.3-YFP.

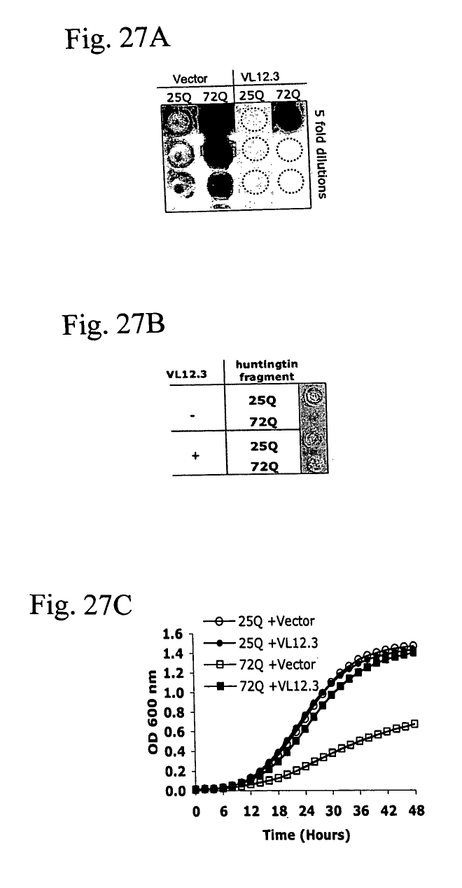

The aggregation state of huntingtin in the presence and absence of V_{L}12.3-YFP was measured eight hours post-induction by a filter retardation assay. This assay consists of lysing cells and passing the lysate through a filter with 0.2 μm pores, trapping aggregates. The amount of aggregated httex1Q72-CFP is then visualized by CFP fluorescence. As shown in FIG. 27A, cells expressing the intrabody had much less aggregated httex1Q72-CFP. This result was confirmed by fluorescence microscopy; expression of V_{L}12.3-YFP resulted in significantly reduced aggregation when measured by this method as well.

Finally, we tested the ability of the intrabody to inhibit HD related cytotoxicity in yeast. S. cerevisiae expressing huntingtin with long polyglutamine tracts have been shown to grow slower than those expressing huntingtin with shorter polyglutamine tracts (Meriin, A. B. et al., J Cell Biol 157: 997-1004, 2002). Growth assays were performed on the cell lines mentioned above. The cell line expressing both V_{L}12.3-YFP and httex1Q72-CFP grew at a significantly faster rate than that which expressed the empty vector and httex1Q72-CFP, as demonstrated by a spotting assay in which the cells were plated on solid media (FIG. 27B). Growth curves were also collected by measuring the optical density (OD) of cultures at 600 nM as a function of time (FIG. 27C). The inhibition of aggregation and toxicity observed in the yeast system upon expression of V_{L}12.3 suggests that a conserved mechanism for htt toxicity is conserved in mammalian and yeast HD models. This confirms the value of S. cerevisiae models in screening and testing potential therapeutic molecules.

Discussion

We have developed a highly potent intracellular antibody against the N-terminal 20 amino acids of the huntingtin protein, htt, which is mutated in Huntington's Disease (HD) and forms intracellular aggregates in medium spiny neurons of the striatum. This new intracellular antibody, V_{L}12.3, efficiently prevents the aggregation and toxicity of htt-exon1 and may therefore be useful in treatment of HD by gene therapy. We removed the 15 disulfide bond of the single-domain antibody, V_{L} (1), by site-directed mutagenesis in order to make its properties, such as stability and affinity, independent of the oxidation state of its environment. We next greatly improved the binding affinity of the antibody by mutagenesis and screening for improved binding. In comparison to previously described intrabodies against htt (Colby, D. W. et al., J Mol Biol 342: 901-912, 2004; Khoshnan, A., et al., Proc Natl Acad Sci U S A 99: 1002-1007, 2002; Lecerf, J. M. et al., Proc Natl Acad Sci U S A 98: 4764-4769, 2001), this intrabody effectively prevented aggregation at 10-fold lower expression levels or plasmid ratios, and was able to reduce intracellular aggregation of mutant htt-exon1 protein almost completely. Given the relative inefficiency of viral gene delivery to the central nervous system, it is essential that in any proposed gene therapy, the therapeutic protein whose gene is delivered should work as efficiently as possible. For this reason, V_{L}12.3 may prove useful in treating HD through gene therapy, in addition to use as a research tool in further studies of the role of htt aggregation in HD pathogenesis.

In a cell-based assay, we explored the ability of V_{L}12.3 to eliminate intracellular aggregates of mutant htt-exon1. Recently there has been some discussion of the role that htt aggregates and aggregation might play in HD (Schaffar, G. et al., Mol Cell 15: 95-105, 2004). We used the formation of large inclusions in the presence of overexpressed htt exon1 as a measure of intrabody potency, although smaller intermediates in the aggregation process may be responsible for toxicity, or other abnormal protein interactions involving misfolded htt-exon1 may be involved. It is therefore noteworthy that when V_{L}12.3 was expressed along with httex1Q97-GFP, greater than 90% of both aggregation and cell toxicity were prevented.

This study also illustrates the impact of disulfide bond formation (or lack thereof) in the cytoplasm on intracellular binding affinity in intrabody-antigen interactions. Conventional wisdom suggests that disulfide bonds do not form in the cytoplasm. However, disulfide bond formation has been observed following oxidative stress (Cumming, R. C. et al., J Biol Chem 279: 21749-21758, 2004), fueling debate within the intrabody research community about whether such bonds form in cytoplasmically expressed antibody fragments. We found a dramatic lowering of the in vitro affinity when the cysteines were replaced by the hydrophobic residues alanine and valine (FIG. 24B). However, these mutations did not alter intracellular intrabody potency, as measured when the intrabody was present at a high plasmid ratio (FIG. 24C). This strongly implies that the disulfide bond does not form even when the cysteine residues are present in this case, given the dramatic effect of cysteine mutation on in vitro affinity. It is also interesting to note that mutation of the cysteine residues did not significantly alter antibody expression (on the yeast surface in this case, FIG. 24A) in contrast to other published reports (Graff, C. P. et al., Protein Eng Des Sel 17: 293-304, 2004; Ramm, K. et al., J Mol Biol 290: 535-546, 1999).

Several reports have brought into question the relevance of antibody affinity in predicting efficacy of intracellular antibodies (Rajpal, A. et al., J Biol Chem 276: 33139-33146, 2001; Arafat, W. et al., Cancer Gene Ther 7: 1250-1256, 2000), suggesting that only expression levels are relevant. However, V_{L} is expressed at levels equivalent to or even above V_{L}12.3 (FIG. 25H). Therefore, affinity is clearly a key determinant in intrabody efficacy in the present case, consistent with the equilibrium relationship:

$$\frac{[ Intrabody \cdot Antigen ]}{[ Antigen ]} = \frac{[ Intrabody ]}{Kd} \qquad ( 1 )$$

where [Intrabody·Antigen] is the concentration of the bound complex. From this relationship, it is clear that high level intrabody overexpression can at least partially compensate for diminished intracellular affinity, as we demonstrate here for the wild-type V_{L} intrabody and V_{L},C22V, C89A. For a micromolar-affinity intrabody, however, micromolar expression levels are necessary even when antigen concentration is much lower than micromolar, as it is likely to be in striatal neurons in vivo. V_{L}12.3, with 3 nM affinity, should be effective at nanomolar level concentrations. In earlier reports, the role of affinity was obscured by measuring antibody affinity in oxidizing (extracellular) environments, where disulfide bonds will form, for comparison to intracellular assays for activity, in which disulfide bonds are unlikely to form. By mutating the cysteines of V_{L} so that no disulfide bond will form, we assessed the protein's properties (and improved its affinity) under oxidizing, extracellular conditions while maintaining the structurally relevant cytoplasmic form.

We are working to assess whether the V_{L}12.3 intrabody binds to wild-type htt in HD heterozygotes, and whether it alters wild-type function. The function or functions of wt-htt are still being investigated and are not conclusively known at present. However, co-transfection of V_{L}12.3 with httex1Q97-GFP did not decrease httex1Q97-GFP expression levels. Also, the precise binding epitope within the first 20 amino acids recognized by V_{L}12.3 is also unknown, and subtle changes in the epitope may have occurred during affinity maturation.

Single-domain intrabodies without disulfide bonds, such as V_{L}12.3, are a minimal and versatile unit for antigen recognition. Single-domain antibodies (Holt, L. J. et al., Trends Biotechnol 21: 484-490, 2003) and structurally analogous domains (Xu, L. et al., Chem Biol 9: 933-942, 2002) are increasingly being exploited as alternatives to single chain antibodies for molecular recognition. The approach demonstrated has application in engineering existing intrabodies for increased potency against other disease targets, including Parkinson's disease, HIV, and cancer.

### Example 6

Functional Delivery of Intrabody V_{L}12.3 into Mammalian Cells Using a Virus.

A strain of adenovirus (Ad-V_{L}12.3) was created that carries the V_{L}12.3 gene. V_{L}12.3 was subcloned into the transfer vector pACCMV2, and the University of Michigan Vector Core facility (www.med.umich.edu/vcore/) produced the engineered virus.

An established neuronal cell model of HD (Apostol B L, et al, Proc Natl Acad Sci U S A. May 13, 2003) was used to asses the function of the virus. Cells were treated with viral lysates (estimated MOI of 100), or were not treated (negative control). Twenty-four hours post-infection, expression of the huntingtinQ103-GFP transgene was induced using 500 nM muristerone A (Invitrogen). After an additional twenty-four hours, the aggregation state of huntingtinQ103-GFP was observed using fluorescence microscopy.

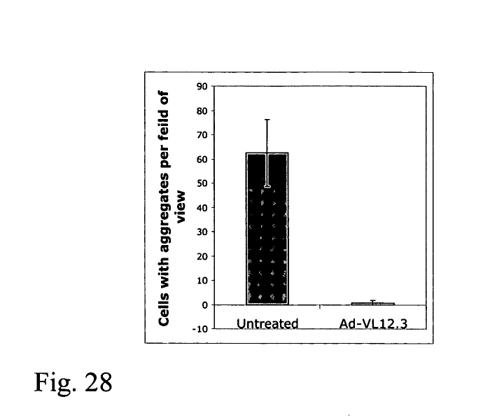

Cells that had been exposed to the Ad-V_{L}12.3 virus were significantly less likely to contain aggregates than cells which had not received treatment, as shown in FIG. 28. Samples imaged demonstrated aggregation in the untreated cells expressing huntingtin Q103-GFP, but aggregation was not detected in the AD-V_{L}12.3-treated cells.

Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. An isolated disulfide-independent, single-domain antibody or antigen-binding fragment thereof.

2. The isolated antibody or antigen-binding fragment thereof of claim 1, wherein the antibody or antigen-binding fragment thereof is disulfide-free.

3. The isolated antibody or antigen-binding fragment thereof of claim 1, wherein the antibody affinity is between about 50 nM and about 5 nM.

4. The isolated antibody or antigen-binding fragment thereof of claim 1, wherein the antibody affinity is at least about 10 nM.

5. The isolated antibody or antigen-binding fragment thereof of claim 1, wherein the antibody or antigen-binding fragment thereof is linked to a targeting molecule.

6. The isolated antibody or antigen-binding fragment thereof of claim 5, wherein the targeting polypeptide is a nuclear localization sequence (NLS).

7. The isolated antibody of claim 5, wherein the targeting molecule's target is a neuronal cell.

8. The isolated antibody or antigen-binding fragment thereof of claim 1, wherein the antibody or antigen-binding fragment thereof is linked to a protein transduction domain (PTD).

9. The isolated antibody or antigen-binding fragment thereof of claim 8, wherein the PTD is selected from the group consisting of: a TAT protein, antennepedia protein, and synthetic poly-arginine.

10. The isolated antibody or antigen-binding fragment thereof of claim 1, wherein the antibody or antigen-binding fragment thereof is linked to a reporter polypeptide.

11. The isolated antibody or antigen-binding fragment thereof of claim 10, wherein the reporter polypeptide is selected from the group consisting of yellow fluorescent protein (YFP), cyan fluorescent protein (CFP), β-galactosidase, chloramphenicol acetyl transferase (CAT), luciferase, green fluorescent protein (GFP).

12. The isolated antibody or antigen-binding fragment thereof of claim 1, wherein the antibody or antigen-binding fragment thereof comprises a single light chain polypeptide comprising the amino acid sequence set forth as SEQ ID NO: 1 or SEQ ID NO:2.

13. The isolated antibody or antigen-binding fragment thereof of claim 1, wherein the antibody or antigen-binding fragment thereof comprises a single light chain polypeptide comprising the amino acid sequence set forth as SEQ ID NO:10.

14. The isolated antibody or antigen-binding fragment thereof of claim 1, wherein the antibody or antigen-binding fragment thereof comprises an amino acid sequence that is a fragment of the amino acid sequence set forth as SEQ ID NO: 3 or SEQ ID NO:4.

15. The isolated antibody or antigen-binding fragment thereof of claim 1, wherein the antibody or antigen-binding fragment thereof inhibits huntingtin aggregation.

16. The isolated antibody or antigen-binding fragment thereof of claim 15, wherein the antibody or antigen-binding fragment thereof specifically binds the N-terminus of huntingtin protein.

17. The isolated antibody or antigen-binding fragment thereof of claim 16 wherein the antibody specifically binds the region of the N-terminus encoded by exon 1 of the huntingtin gene.

18. An isolated antibody or antigen-binding fragment thereof that specifically binds to an epitope on huntingtin protein, wherein the antibody comprises a single light chain polypeptide comprising the amino acid sequence set forth as SEQ ID NO: 1, SEQ ID NO:2, or SEQ ID NO:10.

19-21. (canceled)

22. The isolated antibody or antigen-binding fragment thereof of claim 18, wherein the antibody or antigen-binding fragment thereof is a disulfide-independent antibody.

23-43. (canceled)

44. A method of making a disulfide-independent single-domain antibody comprising:
obtaining a single-domain antibody that specifically binds to a target protein,
mutating at least one or more cysteine amino acids in the antibody, wherein the cysteine mutation removes one or more disulfide bonds from the antibody,
applying directed evolution to the amino acid sequence of the antibody, and
contacting the directly evolved antibody with the target protein to determine specific binding of the directly evolved antibody to the target protein, wherein the directly evolved antibody is a disulfide-independent single domain antibody.

45-74. (canceled)

75. A method of preventing or treating a disease in a subject comprising:
administering the antibody or antigen-binding fragment thereof of claim 1 to a subject in need of such treatment in an amount effective to prevent or treat the disease in the subject.

76-122. (canceled)

123. A method of inhibiting aggregation of huntingtin protein in a cell comprising, contacting the cell intracellularly with the antibody or antigen-binding fragment of claim 18 in an amount effective to inhibit huntingtin aggregation in the cell.

124-131. (canceled)

132. A method of diagnosing a disease or disorder in a subject comprising:
contacting a sample obtained from a subject with a disulfide-independent, single-domain antibody or antigen-binding fragment thereof of claim 1,
determining a level of the protein to which the disulfide-independent, single-domain antibody or antigen binding fragment thereof specifically binds,
comparing the level obtained to a control level, wherein a difference in the level obtained and the control level is diagnostic for the disease or disorder in the subject.

133-145. (canceled)


SEQUENCE LISTING (10 sequences)

SEQ ID NO: 1 - protein, 111 aa, Artificial Sequence
  Synthetic Peptide
QPVLTQSPSVSAAPRQRVTISCSGSNSNIGSNTVNWFQQLPGRAPELLMYYDDLLAPGVSDRFSGSKSGTSASLAISGLQSEDEADYYCATWDDSLNGWVFGGGTKVTVLS

SEQ ID NO: 2 - protein, 112 aa, Artificial Sequence
  Synthetic Peptide
SRPVLTQSPSVSAAPRQRVTISVSGSNSNIGSNTVNWIQQLPGRAPELLMYDDDLLAPGVSDRFSGSRSGTSASLTISGLQSEDEADYYAATWDDSLNDWVFGGGTKVTVLS

SEQ ID NO: 3 - protein, 267 aa, Homo sapiens
SASQVQLVKSEAEVKKPGSSVRVSCKASGGTISSCAISWVRQAPGQGLEWMGGIIPMFDTTNYAQNFQGRVTITADESTSTAYMDLSSLRSEDTAVYYCARTYYHDTSDNDGTYGMDVWGQGTTVTVSSASTKGPSGILGSGGGGSGGGGSGGGGSQPVLTQSPSASGTPGQRVTISCSGSTSNIGNNAVNWFQQFPGKAPKLLVYYDDLLPSGVSDRFSGSKSGTSASLAISGLQSEDEADYYCATWDDSLNGWVFGGGTKVTVLS

SEQ ID NO: 4 - protein, 267 aa, Homo sapiens
SASQVQLVESEAEVKKPGSSVRVSCKASGGTISSCAISWVRQAPGQGLEWMGGIIPMFDTTNYAQNFQGRVTITADESTSTAYMDLSSLRSEDTAVYYCARTYYHDTRDNDGTYGMDVWGQGTTVTVSSASTKGPSGILGSGGGGSGGGGSGGGGSQPVLTQSPSASGTPGQRVTISCSGSSSNIGSNTVNWFQQLPGTAPELLMYYDDLLASGVSDRFSGSKSGTSASLAISGLQSEDEGDYYCASWDDNLNGWVFGGGTKLTVLS

SEQ ID NO: 5 - DNA, 50 bp, Artificial sequence
  Synthetic Oligonucleotide
cgacgattgaaggtagatacccatacgacgttccagactacgctctgcag

SEQ ID NO: 6 - DNA, 47 bp, Artificial Sequence
  Synthetic Oligonucleotide
cagatctcgagctattacaagtcttcttcagaaataagcttttgttc

SEQ ID NO: 7 - DNA, 22 bp, Artificial Sequence
  Synthetic Oligonucleotide
gttccagactacgctctgcagg

SEQ ID NO: 8 - DNA, 24 bp, Artificial sequence
  Synthetic Oligonucleotide
gattttgttacatctacactgttg

SEQ ID NO: 9 - protein, 20 aa, Homo sapiens
MATLEKLMKAFESLKSFQQQ

SEQ ID NO: 10 - protein, 121 aa, Artificial Sequence
  Synthetic Peptide
MGSQPVLTQSPSVSAAPRQRVTISVSGSNSNIGSNTVNWIQQLPGRAPELLMYDDDLLAPGVSDRFSGSRSGTSASLTISGLQSEDEADYYAATWDDSLNGWVFGGGTKVTVLSGHHHHHH